(19) United States
(12) Patent Application Publication
(10) Pub. No.: US 2026/0166307 A1
(43) Pub. Date: Jun. 18, 2026

(54) LABOR SPLITTING ARRANGEMENTS

(71) Applicant: COCHLEAR LIMITED, Macquarie University, NSW (AU)

(72) Inventors: Jowan PITTEVILS, Macquarie University (AU); Werner MESKENS, Macquarie University (AU)

(21) Appl. No.: 19/127,515

(22) PCT Filed: Nov. 7, 2023
(86) PCT No.: PCT/IB2023/061237

§ 371 Date: May 6, 2025

Related U.S. Application Data
(60) Provisional application No. 63/423,391, filed on Nov. 7, 2022.

(51) Int. Cl. A61N 1/05; H04R 25/00

(52) U.S. Cl. CPC A61N 1/0541; H04R 25/552; H04R 25/70

(57) ABSTRACT

A system, including a first device and a second device, wherein the first device is a component of a sensory prosthesis configured to receive a data stream and evoke a sensory percept based on the data stream, and the second device is configured to provide spatial output to the first device and/or another device remote from the second device.

### CROSS-REFERENCE TO RELATED APPLICATIONS

This application claims priority to U.S. Provisional Application No. 63/423,391, entitled LABOR SPLITTING ARRANGEMENTS, filed on Nov. 7, 2022, naming Jowan PITTEVILS as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a system, comprising a first device and a second device, wherein the first device is a component of a sensory prosthesis configured to receive a data stream and evoke a sensory percept based on the data stream, and the second device is configured to provide spatial output to the first device and/or another device remote from the second device.

In an embodiment, there is a method, comprising at least one of receiving a first wireless signal or sending second wireless signal by a first device, receiving at a second device a data stream, wherein the second device is a component of a sensory prosthesis, and transmitting by the second device to the first device data based on the data stream, wherein at least one of: (1) a receiver and/or transceiver of the second device is adjusted based on data based on the first wireless signal, which receiver and/or transceiver receives the data stream; or (2) a transmitter and/or transceiver of another device is adjusted based on data based on the second wireless signal, wherein the transmitter and/or transceiver transmits the data stream.

In another exemplary embodiment, there is a system, comprising a first device and a second device, wherein the system is a sensory supplement system, a communication load of the system is split between the first device and the second device, and at least one of the first device or the second device is configured to be one of worn on or implanted in a recipient of the system.

In another exemplary embodiment, there is a method, comprising at least one of: receiving a first wireless signal, transmitting a second wireless signal or capturing sound by a first device and receiving at a second device a data stream, wherein one of the first device or the second device is an implanted device implanted in a recipient and the other of the first device or the second device is an external device external to the recipient, the implanted device includes circuitry on which resides a first portion of a software stack, the external device includes circuitry on which resides a second portion of a software stack, and the method comprises evoking a sensory percept via a process that runs the first portion on the implanted device and runs the second portion on the external device.

In another embodiment, there is a hearing system, comprising a first hearing prosthesis including a sound processor, a microphone, and a stimulator and a second hearing prosthesis including a sound processor, a microphone and a stimulator, wherein the first hearing prosthesis includes a receiver and/or transceiver configured to receive a data stream, the first hearing prosthesis is configured to evoke a hearing percept based on the data stream using the stimulator, and the second hearing prosthesis is configured to provide spatial output to the first hearing prosthesis and/or another device remote from the second hearing prosthesis.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIG. 1B is a top view of an exemplary hearing prosthesis in which at least some of the teachings detailed herein are applicable;

FIG. 1C is a side view of an exemplary hearing prosthesis in which at least some of the teachings detailed herein are applicable;

FIG. 1D is a view of an exemplary sight prosthesis in which at least some of the teachings herein are applicable;

FIG. 1E presents an exemplary external component that provides a baseline for an exemplary external component that is utilized with the teachings herein;

FIGS. 2A-B are exemplary functional block diagrams of a prosthesis that provides a baseline for the inventive teachings herein;

FIG. 2C presents an exemplary external component that provides a baseline for an exemplary external component that is utilized with the teachings herein;

FIGS. 3A-3C are exemplary functional block diagrams of cochlear implants that provides a baseline for the inventive teachings herein;

FIG. 4A is a simplified schematic diagram of a transceiver unit of an external device that provides a baseline for the inventive teachings herein;

FIG. 4B is a simplified schematic diagram of a transmitter unit of an external device that provides a baseline for the inventive teachings herein;

FIG. 4C is a simplified schematic diagram of a stimulator/receiver unit including a data receiver of an implantable device that provides a baseline for the inventive teachings herein;

FIG. 4D is a simplified schematic diagram of a stimulator/receiver unit including a data transceiver of an implantable device that provides a baseline for the inventive teachings herein;

FIG. 4E is a simplified schematic diagram of a stimulator/receiver unit including a data receiver and a communication component configured to vary the effective coil area of an implantable device that provides a baseline for the inventive teachings herein;

FIG. 4F is a simplified schematic diagram of a stimulator/receiver unit including a data transceiver and a communication component that provides a baseline for the inventive teachings herein;

FIGS. 5 and 6 and 7 and 7A and 8 and 9 and 10 present exemplary component that provides a baseline for the inventive teachings herein;

FIGS. 11 and 12 and 15A and 15B and 15C provide exemplary scenarios of use of some embodiments and/or systems of some embodiments;

FIG. 13 provides an exemplary schematic of an exemplary system;

FIG. 14 provides an exemplary flowchart for an exemplary method;

FIG. 16 provides an exemplary flowchart for an exemplary method; and

FIG. 17 provides an exemplary flowchart for an exemplary method.

### DETAILED DESCRIPTION

Merely for ease of description, the techniques presented herein are primarily described herein with reference to an illustrative medical device, namely a hearing prosthesis. First introduced is a cochlear implant. The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant or a conventional acoustic hearing aid, corresponds to a disclosure of another embodiment of using such teaching with, at least in conjunction with, another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, retinal implants, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

Embodiments are also applicable to conventional hearing aids.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

The techniques presented herein are also described with reference by way of background to another illustrative medical device, namely a retinal implant. As noted above, the techniques presented herein are also applicable to the technology of vestibular devices (e.g., vestibular implants), visual devices (i.e., bionic eyes), as well as sensors, pacemakers, drug delivery systems, defibrillators, functional electrical stimulation devices, catheters, seizure devices (e.g., devices for monitoring and/or treating epileptic events), sleep apnea devices, electroporation, etc.

Any reference to one of the above-noted sensory prostheses corresponds to an alternate disclosure using one of the other above-noted sensory prostheses unless otherwise noted providing that the art enables such.

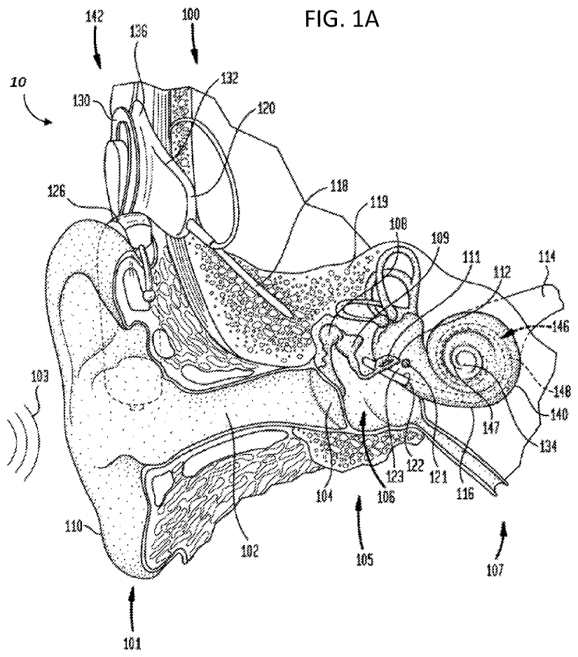

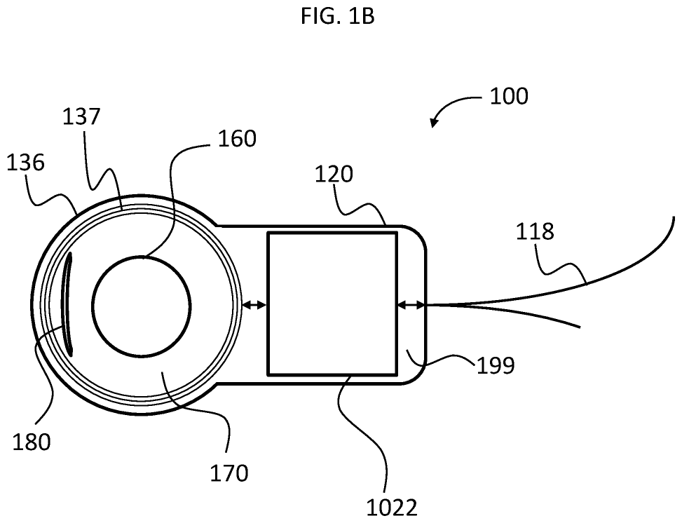

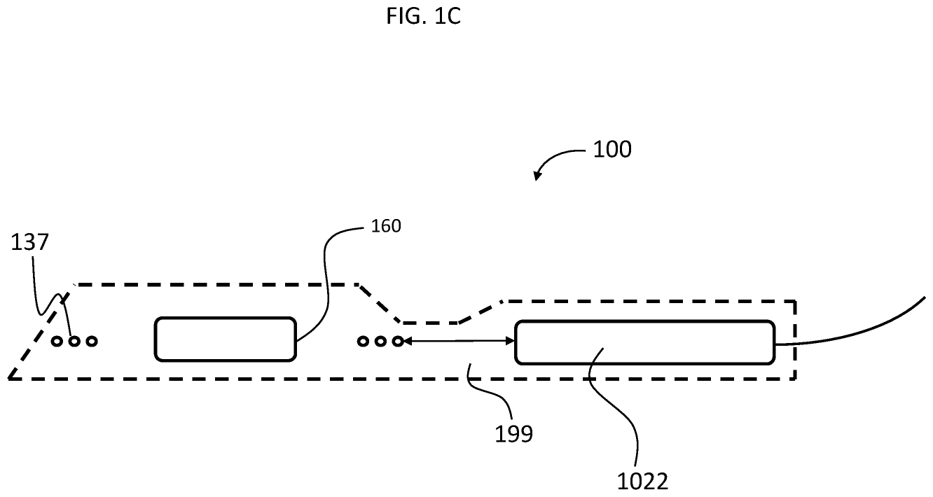

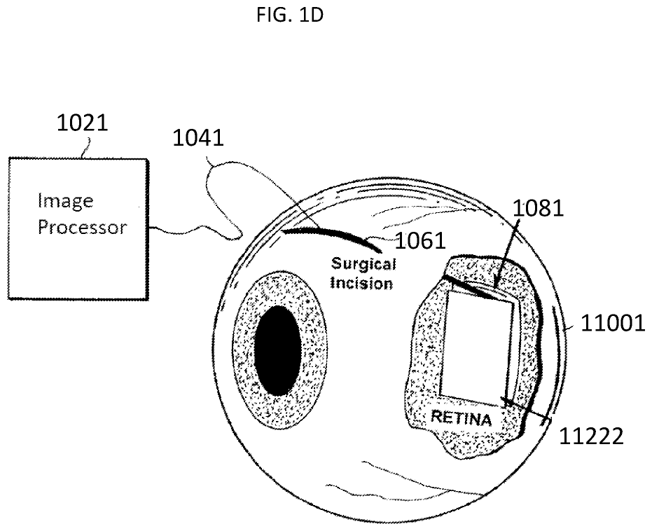

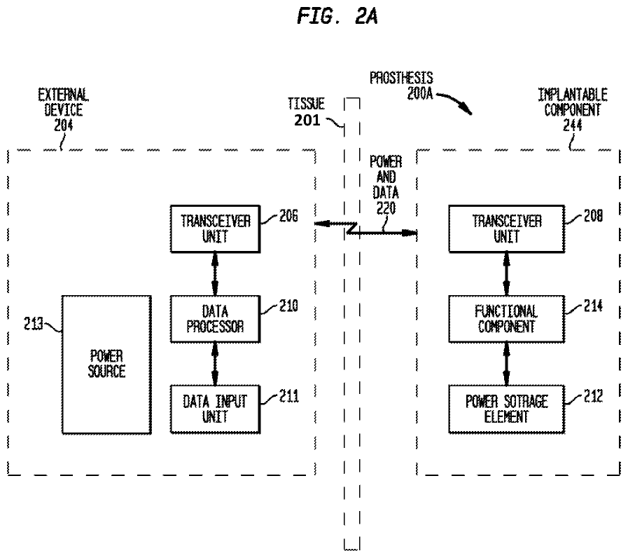

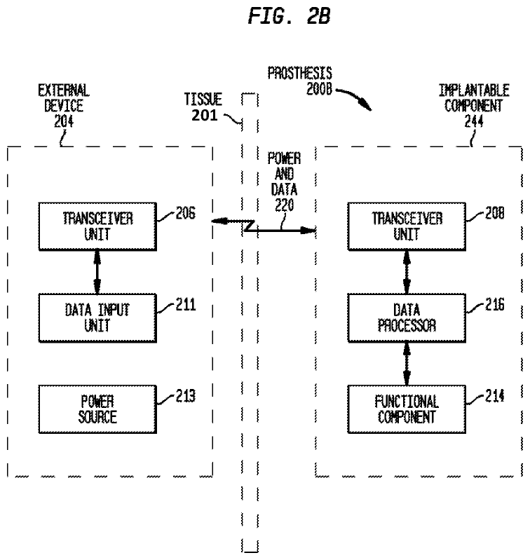

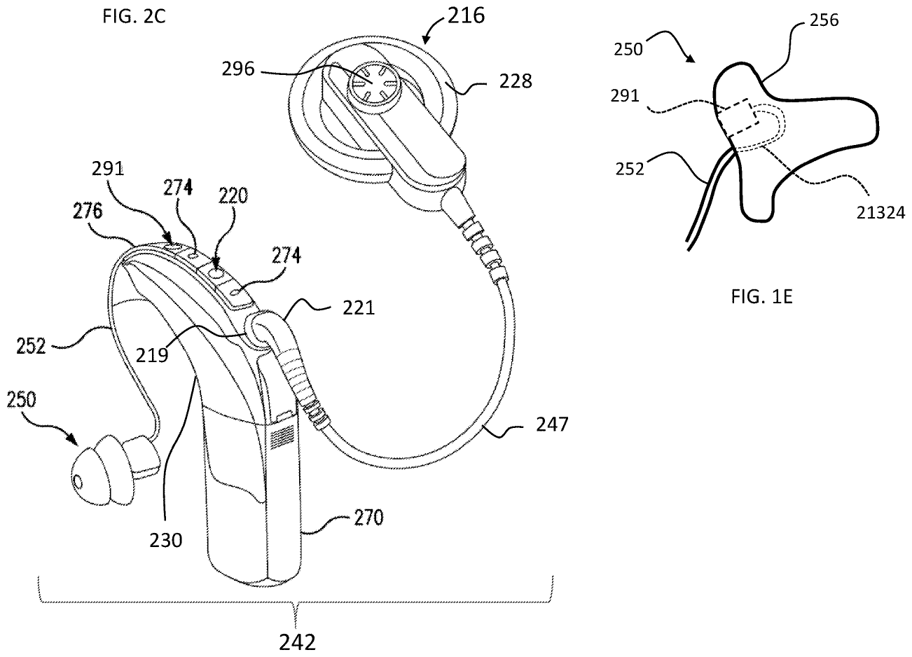

FIG. 1A is a perspective view of a cochlear implant, referred to as cochlear implant 100, implanted in a recipient, to which some embodiments detailed herein and/or variations thereof are applicable. The cochlear implant 100 is part of a sensory supplement system 10, here, a cochlear implant system 10, or a hearing prosthesis 10, that can include external components in some embodiments, as will be detailed below. It is noted that the teachings detailed herein are applicable, in at least some embodiments, to partially implantable and/or totally implantable cochlear implants (i.e., with regard to the latter, such as those having an implanted microphone). It is further noted that the teachings detailed herein are also applicable to other stimulating devices that utilize an electrical current beyond cochlear implants (e.g., auditory brain stimulators, pacemakers, etc.). Additionally, it is noted that the teachings detailed herein are also applicable to other types of hearing prostheses, such as by way of example only and not by way of limitation, bone conduction devices, direct acoustic cochlear stimulators, middle ear implants, etc. Indeed, it is noted that the teachings detailed herein are also applicable to so-called hybrid devices. In an exemplary embodiment, these hybrid devices apply both electrical stimulation and acoustic stimulation to the recipient. Any type of hearing prosthesis to which the teachings detailed herein and/or variations thereof that can have utility can be used in some embodiments of the teachings detailed herein. The teachings herein are also applicable to conventional acoustic hearing aids.

In view of the above, it is to be understood that at least some embodiments detailed herein and/or variations thereof are directed towards a body-worn sensory supplement medical device (e.g., the hearing prosthesis of FIG. 1A, which supplements the hearing sense, even in instances where all natural hearing capabilities have been lost). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities remaining and to recipients having no natural vision capabilities remaining). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant, and where the implanted cochlear implant includes a battery, that is recharged by the power provided from the external device 142.

In the illustrative arrangement of FIG. 1A, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1A, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with the teachings herein.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil assembly 137. Internal coil assembly 137 typically includes a wire antenna coil comprised of multiple turns of electrically insulated single-strand or multi-strand platinum or gold wire, as will be described in greater detail below.

Cochlear implant 100 further comprises a main implantable component 120 and an elongate electrode assembly 118. Collectively, the coil assembly 137, the main implantable component 120, and the electrode assembly 118 correspond to the implantable component of the system 10.

In some embodiments, internal energy transfer assembly 132 and main implantable component 120 are hermetically sealed within a biocompatible housing or within the device in general (the housing per se may not be hermetically sealed). In some embodiments, main implantable component 120 includes an implantable microphone assembly (not shown) and a sound processing unit (not shown) to convert the sound signals received by the implantable microphone or via internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown in FIG. 1A) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123, or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

FIG. 1B depicts an exemplary high-level diagram of the implantable component 100 of the system 10, looking downward from outside the skull towards the skull. As can be seen, implantable component 100 includes a magnet 160 that is surrounded by a coil 137 that is in two-way communication (although in some instances, the communication is one-way) with a receiver stimulator unit 1022, which in turn is in communication with the electrode assembly 118.

Still with reference to FIG. 1B, it is noted that the receiver stimulator unit 1022, and the magnet apparatus 160 are located in a housing made of an elastomeric material 199, such as by way of example only and not by way of limitation, silicone. Hereinafter, the elastomeric material 199 of the housing will be often referred to as silicone. However, it is noted that any reference to silicone herein also corresponds to a reference to any other type of component that will enable the teachings detailed herein and/or variations thereof, such as, by way of example and not by way of limitation only, bio-compatible rubber, etc.

As can be seen in FIG. 1B, the housing made of elastomeric material 199 includes a slit 180 (not shown in FIG. 1C, as, in some instances, the slit is not utilized). In some variations, the slit 180 has utilitarian value in that it can enable insertion and/or removal of the magnet apparatus 160 from the housing made of elastomeric material 199.

It is noted that magnet apparatus 160 is presented in a conceptual manner. In this regard, it is noted that in at least some instances, the magnet apparatus 160 is an assembly that includes a magnet surrounded by a biocompatible coating. Still further by way of example, magnet apparatus 160 is an assembly where the magnet is located within a container having interior dimensions generally corresponding to the exterior dimensions of the magnet. This container can be hermetically sealed, thus isolating the magnet in the container from body fluids of the recipient that penetrate the housing (the same principle of operation occurs with respect to the aforementioned coated magnet). In an exemplary embodiment, this container permits the magnet to revolve or otherwise move relative to the container. Additional details of the container will be described below. In this regard, it is noted that while sometimes the term magnet is used as shorthand for the phrase magnet apparatus, and thus any disclosure herein with respect to a magnet also corresponds to a disclosure of a magnet apparatus according to the aforementioned embodiments and/or variations thereof and/or any other configuration that can have utilitarian value according to the teachings detailed herein.

Briefly, it is noted that there is utilitarian value with respect to enabling the magnet to revolve within the container or otherwise move. In this regard, in an exemplary embodiment, when the magnet is introduced to an external magnetic field, such as in an MRI machine, the magnet can revolve or otherwise move to substantially align with the external magnetic field. In an exemplary embodiment, this alignment can reduce or otherwise eliminate the torque on the magnet, thus reducing discomfort and/or reducing the likelihood that the implantable component will be moved during the MRI procedure (potentially requiring surgery to place the implantable component at its intended location) and thus reduce and/or eliminate the demagnetization of the magnet.

Element 136 can be considered a housing of the coil, in that it is part of the housing 199.

With reference now to FIG. 1C, it is noted that the outlines of the housing made from elastomeric material 199 are presented in dashed line format for ease of discussion. In an exemplary embodiment, silicone or some other elastomeric material fills the interior within the dashed line, other than the other components of the implantable device (e.g., plates, magnet, stimulator, etc.). That said, in an alternative embodiment, silicone or some other elastomeric material substantially fills the interior within the dashed lines other than the components of the implantable device (e.g., there can be pockets within the dashed line in which no components and no silicone are located).

It is noted that FIGS. 1B and 1C are conceptual FIGs. presented for purposes of discussion. Commercial embodiments corresponding to these FIGs. can be different from that depicted in the figures.

FIG. 1D presents an exemplary embodiment of a neural prosthesis in general, and a retinal prosthesis and an environment of use thereof, in particular. In some embodiments of a retinal prosthesis, a retinal prosthesis sensor-stimulator 108 is positioned proximate the retina 1101. In an exemplary embodiment, photons entering the eye are absorbed by a microelectronic array of the sensor-stimulator 108 that is hybridized to a glass piece 11222 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 1021 is in signal communication with the sensor-stimulator 1081 via cable 1041 which extends through surgical incision 1061 through the eye wall (although in other embodiments, the image processor 1021 is in wireless communication with the sensor-stimulator 1081). In an exemplary embodiment, the image processor 1021 is analogous to the sound processor/signal processors of the auditory prostheses detailed herein, and in this regard, any disclosure of the latter herein corresponds to a disclosure of the former in an alternate embodiment. The image processor 1021 processes the input into the sensor-stimulator 1081, and provides control signals back to the sensor-stimulator 1081 so the device can provide processed and output to the optic nerve. That said, in an alternate embodiment, the processing is executed by a component proximate to or integrated with the sensor-stimulator 1081. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 1081 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner. Indeed, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

Returning back to the cochlear implant embodiment, FIG. 2A is a baseline functional block diagram of a prosthesis 200A that presents basic features that are utilized. Prosthesis 200A comprises an implantable component 244 configured to be implanted beneath a recipient's skin or other tissue 201 and an external device 204. For example, implantable component 244 may be implantable component 100 of FIG. 1A, and external device may be the external device 142 of FIG. 1A. Similar to the embodiments described above with reference to FIG. 1A, implantable component 244 comprises a transceiver unit 208 which receives data and power from external device 204. External device 204 transmits power and data 220 via transceiver unit 206 to transceiver unit 208 via a magnetic induction data link 220. As used herein, the term receiver refers to any device or component configured to receive power and/or data such as the receiving portion of a transceiver or a separate component for receiving. The details of transmission of power and data to transceiver unit 208 are provided below. With regard to transceivers, it is noted at this time that while embodiments may utilize transceivers, separate receivers and/or transmitters may be utilized as appropriate. Herein, any disclosure of one corresponds to a disclosure of the other and vice versa.

Implantable component 244 may comprises a power storage element 212 and a functional component 214. Power storage element 212 is configured to store power received by transceiver unit 208, and to distribute power, as needed, to the elements of implantable component 244. Power storage element 212 may comprise, for example, a rechargeable battery 212. An example of a functional component may be a stimulator unit 120 as shown in FIG. 1B.

In certain embodiments, implantable component 244 may comprise a single unit having all components of the implantable component 244 disposed in a common housing. In other embodiments, implantable component 244 comprises a combination of several separate units communicating via wire or wireless connections. For example, power storage element 212 may be a separate unit enclosed in a hermetically sealed device, such as the housing, or the combination of the housing and other components, etc. The implantable magnet apparatus and plates associated therewith may be attached to or otherwise be a part of any of these units, and more than one of these units can include the magnet apparatus and plates according to the teachings detailed herein and/or variations thereof.

In the embodiment depicted in FIG. 2A, external device 204 includes a data processor 210 that receives data from data input unit 211 and processes the received data. The processed data from data processor 210 is transmitted by transceiver unit 206 to transceiver unit 208. In an exemplary embodiment, data processor 210 may be a sound processor, such as the sound processor of FIG. 1A for the cochlear implant thereof, and data input unit 211 may be a microphone of the external device.

FIG. 2B presents an alternate embodiment of the prosthesis 200A of FIG. 2A, identified in FIG. 2B as prosthesis 200B. As may be seen from comparing FIG. 2A to FIG. 2B, the data processor can be located in the external device 204 or can be located in the implantable component 244. In some embodiments, both the external device 204 and the implantable component 244 can include a data processor.

As shown in FIGS. 2A and 2B, external device 204 can include a power source 213. Power from power source 213 can be transmitted by transceiver unit 206 to transceiver unit 208 to provide power to the implantable component 244, as will be described in more detail below.

While not shown in FIGS. 2A and 2B, external device 204 and/or implantable component 244 include respective inductive communication components. These inductive communication components can be connected to transceiver unit 206 and transceiver unit 208, permitting power and data 220 to be transferred between the two units via magnetic induction.

As used herein, an inductive communication component includes both standard induction coils and inductive communication components configured to vary their effective coil areas.

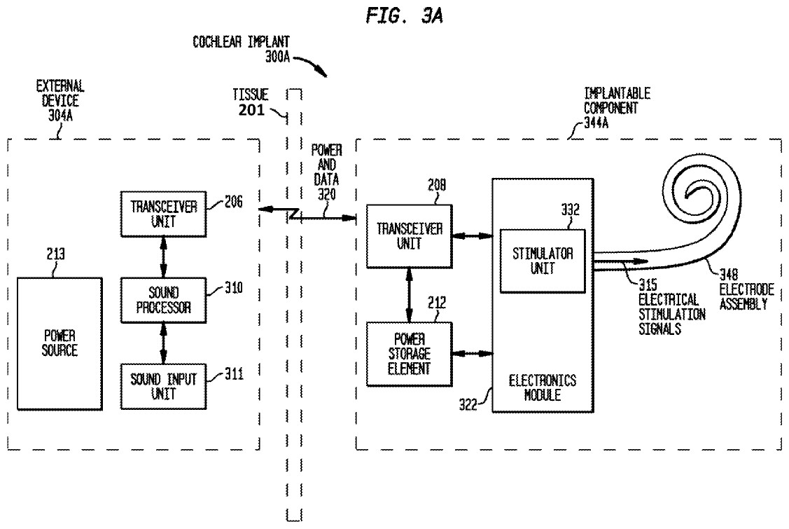

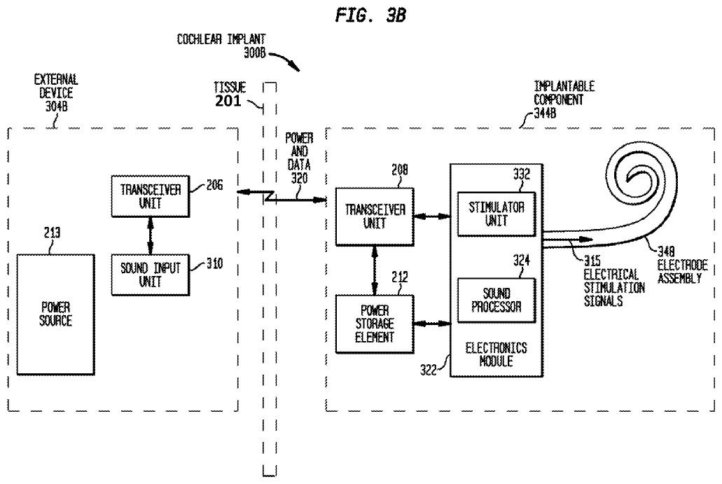

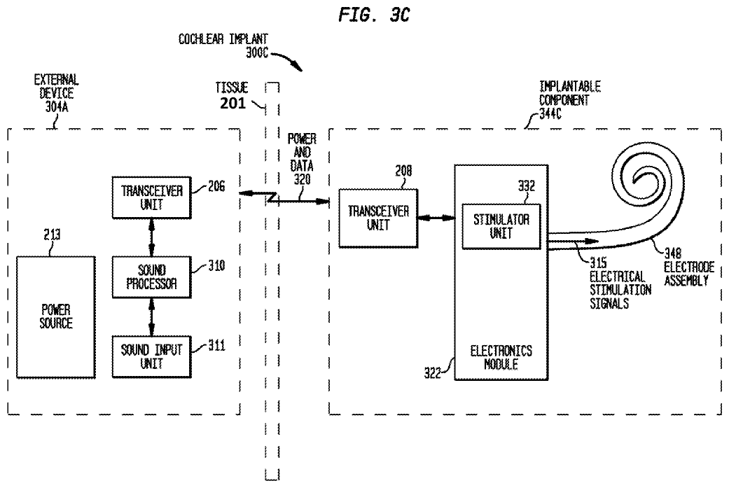

As noted above, prosthesis 200A of FIG. 2A may be a cochlear implant. In this regard, FIG. 3A provides additional details of an embodiment of FIG. 2A where prosthesis 200A is a cochlear implant. Specifically, FIG. 3A is a functional block diagram of a cochlear implant 300.

It is noted that the components detailed in FIGS. 2A and 2B may be identical to the components detailed in FIG. 3A, and the components of 3A may be used in the embodiments depicted in FIGS. 2A and 2B.

Cochlear implant 300A comprises an implantable component 344A (e.g., implantable component 100 of FIG. 1) configured to be implanted beneath a recipient's skin or other tissue 201, and an external device 304A. External device 304A may be an external component such as external component 142 of FIG. 1.

Similar to the embodiments described above with reference to FIGS. 2A and 2B, implantable component 344A comprises a transceiver unit 208 (which may be the same transceiver unit used in FIGS. 2A and 2B) which receives data and power from external device 304A. External device 304A transmits data and/or power 320 to transceiver unit 208 via a magnetic induction data link. This can be done while charging module 212.

Implantable component 344A also comprises a power storage element 212, electronics module 322 (which may include components such as sound processor 126 and/or may include a receiver stimulator unit 332 corresponding to receiver stimulator unit 1022 of FIG. 1B) and an electrode assembly 348 (which may include an array of electrode contacts 148 of FIG. 1A). Power storage element 212 is configured to store power received by transceiver unit 208, and to distribute power, as needed, to the elements of implantable component 344A.

As shown, electronics module 322 includes a stimulator unit 332. Electronics module 322 can also include one or more other functional components used to generate or control delivery of electrical stimulation signals 315 to the recipient. As described above with respect to FIG. 1A, electrode assembly 348 is inserted into the recipient's cochlea and is configured to deliver electrical stimulation signals 315 generated by stimulator unit 332 to the cochlea.

In the embodiment depicted in FIG. 3A, the external device 304A includes a sound processor 310 configured to convert sound signals received from sound input unit 311 (e.g., a microphone, an electrical input for an FM hearing system, etc.) into data signals. In an exemplary embodiment, the sound processor 310 corresponds to data processor 210 of FIG. 2A.

FIG. 3B presents an alternate embodiment of a cochlear implant 300B. The elements of cochlear implant 300B correspond to the elements of cochlear implant 300A, except that external device 304B does not include sound processor 310. Instead, the implantable component 344B includes a sound processor 324, which may correspond to sound processor 310 of FIG. 3A.

As will be described in more detail below, while not shown in the figures, external device 304A/304B and/or implantable component 344A/344B include respective inductive communication components.

FIGS. 3A and 3B illustrate that external device 304A/304B can include a power source 213, which may be the same as power source 213 depicted in FIG. 2A. Power from power source 213 can be transmitted by transceiver unit 306 to transceiver unit 308 to provide power to the implantable component 344A/344B, as will be detailed below. FIGS. 3A and 3B further detail that the implantable component 344A/344B can include a power storage element 212 that stores power received by the implantable component 344 from power source 213. Power storage element 212 may be the same as power storage element 212 of FIG. 2A.

In contrast to the embodiments of FIGS. 3A and 3B, as depicted in FIG. 3C, an embodiment of a cochlear implant 300C includes an implantable component 344C that does not include a power storage element 212. In the embodiment of FIG. 3C, sufficient power is supplied by external device 304A/304B in real time to power implantable component 344C without storing power in a power storage element. In FIG. 3C, all of the elements are the same as FIG. 3A except for the absence of power storage element 212.

Some of the components of FIGS. 3A-3C will now be described in greater detail.

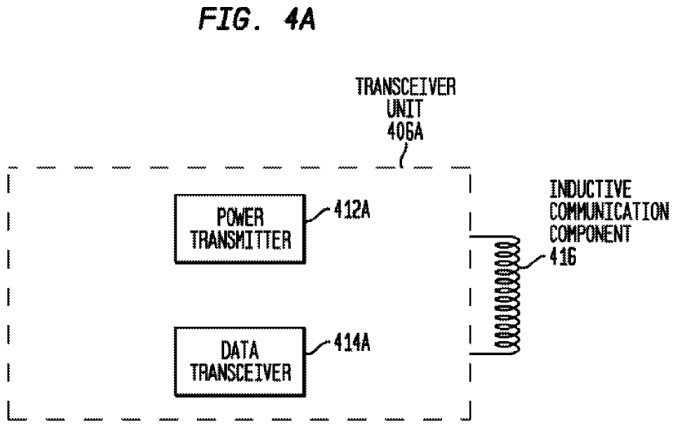

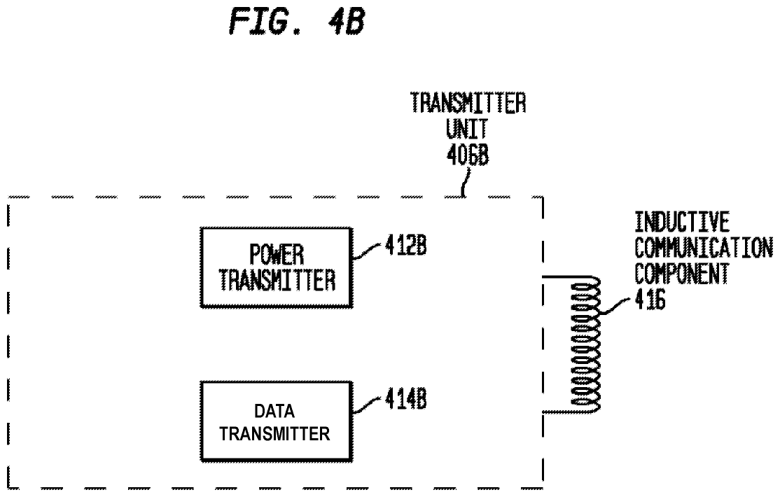

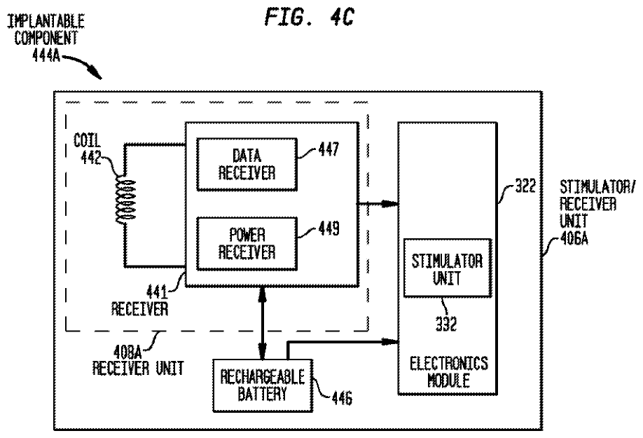

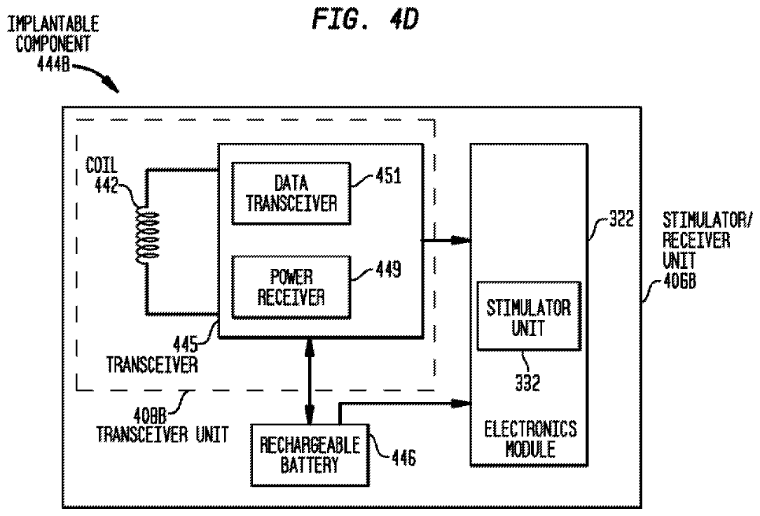

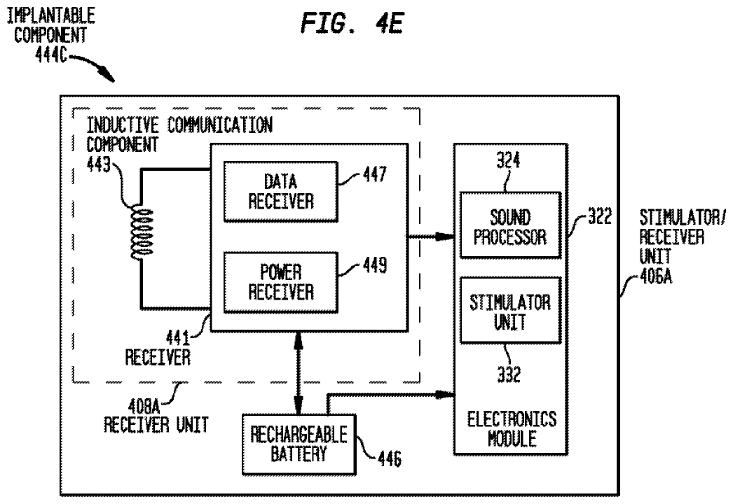

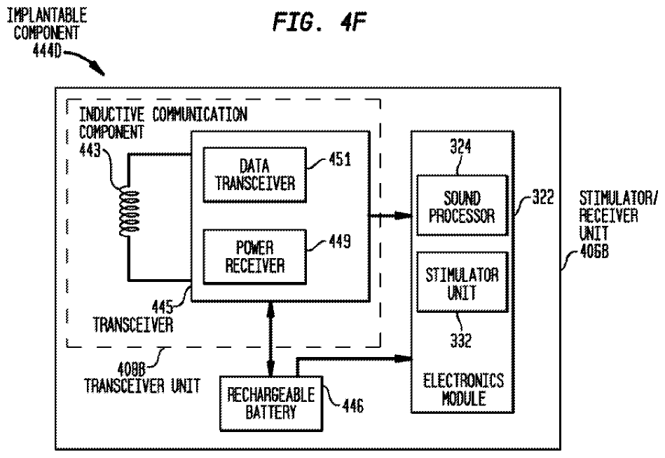

FIG. 4A is a simplified schematic diagram of a transceiver unit 406A in accordance with an embodiment. An exemplary transceiver unit 406A may correspond to transceiver unit 206 of FIGS. 2A-3C. As shown, transceiver unit 406A includes a power transmitter 412 a, a data transceiver 414A and an inductive communication component 416.

In an exemplary embodiment, as will be described in more detail below, inductive communication component 416 comprises one or more wire antenna coils (depending on the embodiment) comprised of multiple turns of electrically insulated single-strand or multi-strand platinum or gold wire (thus corresponding to coil 137 of FIG. 1B). Power transmitter 412A comprises circuit components that inductively transmit power from a power source, such as power source 213, via an inductive communication component 416 to implantable component 344A/B/C (FIGS. 3A-3C). Data transceiver 414A comprises circuit components that cooperate to output data for transmission to implantable component 344A/B/C (FIGS. 3A-3C). Transceiver unit 406A can receive inductively transmitted data from one or more other components of cochlear implant 300A/B/C, such as telemetry or the like from implantable component 344A (FIG. 3A).

Transceiver unit 406A can be included in a device that includes any number of components which transmit data to implantable component 334A/B/C. For example, the transceiver unit 406A may be included in a behind-the-ear (BTE) device having one or more of a microphone or sound processor therein, an in-the-ear device, etc.

FIG. 4B depicts a transmitter unit 406B, which is identical to transceiver unit 406A, except that it includes a power transmitter 412B and a data transmitter 414B.

It is noted that for ease of description, power transmitter 412A and data transceiver 414A/data transmitter 414B are shown separate. However, it should be appreciated that in certain embodiments, at least some of the components of the two devices may be combined into a single device.

FIG. 4C is a simplified schematic diagram of one embodiment of an implantable component 444A that corresponds to implantable component 344A of FIG. 3A, except that transceiver unit 208 is a receiver unit. In this regard, implantable component 444A comprises a receiver unit 408A, a power storage element, shown as rechargeable battery 446, and electronics module 322, corresponding to electronics module 322 of FIG. 3A. Receiver unit 408A includes an inductance coil 442 connected to receiver 441. Receiver 441 comprises circuit components which receive, via an inductive communication component corresponding to an inductance coil 442, inductively transmitted data and power from other components of cochlear implant 300A/B/C, such as from external device 304A/B. The components for receiving data and power are shown in FIG. 4C as data receiver 447 and power receiver 449. For ease of description, data receiver 447 and power receiver 449 are shown separate. However, it should be appreciated that in certain embodiments, at least some of the components of these receivers may be combined into one component.

In the illustrative embodiments, a receiver unit 408A and transceiver unit 406A (or transmitter unit 406B) establish a transcutaneous communication link over which data and power is transferred from transceiver unit 406A (or transmitter unit 406B), to implantable component 444A. As shown, the transcutaneous communication link comprises a magnetic induction link formed by an inductance communication component system that includes inductive communication component 416 and coil 442.

The transcutaneous communication link established by receiver unit 408A and transceiver unit 406A (or whatever other viable component can so establish such a link), in an exemplary embodiment, may use time interleaving of power and data on a single radio frequency (RF) channel or band to transmit the power and data to implantable component 444A. A method of time interleaving power according to an exemplary embodiment uses successive time frames, each having a time length and each divided into two or more time slots. Within each frame, one or more time slots are allocated to power, while one or more time slots are allocated to data. In an exemplary embodiment, the data modulates the RF carrier or signal containing power. In an exemplary embodiment, transceiver unit 406A and transmitter unit 406B are configured to transmit data and power, respectively, to an implantable component, such as implantable component 344A, within their allocated time slots within each frame.

The power received by receiver unit 408A can be provided to rechargeable battery 446 for storage. The power received by receiver unit 408A can also be provided for distribution, as desired, to elements of implantable component 444A. As shown, electronics module 322 includes stimulator unit 332, which in an exemplary embodiment corresponds to stimulator unit 322 of FIGS. 3A-3C, and can also include one or more other functional components used to generate or control delivery of electrical stimulation signals to the recipient.

In an embodiment, implantable component 444A comprises a receiver unit 408A, rechargeable battery 446 and electronics module 322 integrated in a single implantable housing, referred to as stimulator/receiver unit 406A. It would be appreciated that in alternative embodiments, implantable component 344 may comprise a combination of several separate units communicating via wire or wireless connections.

FIG. 4D is a simplified schematic diagram of an alternate embodiment of an implantable component 444B. Implantable component 444B is identical to implantable component 444A of FIG. 4C, except that instead of receiver unit 408A, it includes transceiver unit 408B. Transceiver unit 408B includes transceiver 445 (as opposed to receiver 441 in FIG. 4C). Transceiver unit 445 includes data transceiver 451 (as opposed to data receiver 447 in FIG. 4C).

FIGS. 4E and 4F depict alternate embodiments of the implantable components 444A and 444B depicted in FIGS. 4C and 4D, respectively. In FIGS. 4E and 4F, instead of coil 442, implantable components 444C and 444D (FIGS. 4E and 4F, respectively) include inductive communication component 443. Inductive communication component 443 is configured to vary the effective coil area of the component, and may be used in cochlear implants where the exterior device 304A/B does not include a communication component configured to vary the effective coil area (i.e., the exterior device utilizes a standard inductance coil). In other respects, the implantable components 444C and 444D are substantially the same as implantable components 444A and 444B. Note that in the embodiments depicted in FIGS. 4E and 4F, the implantable components 444C and 444D are depicted as including a sound processor 342. In other embodiments, the implantable components 444C and 444D may not include a sound processor 342.

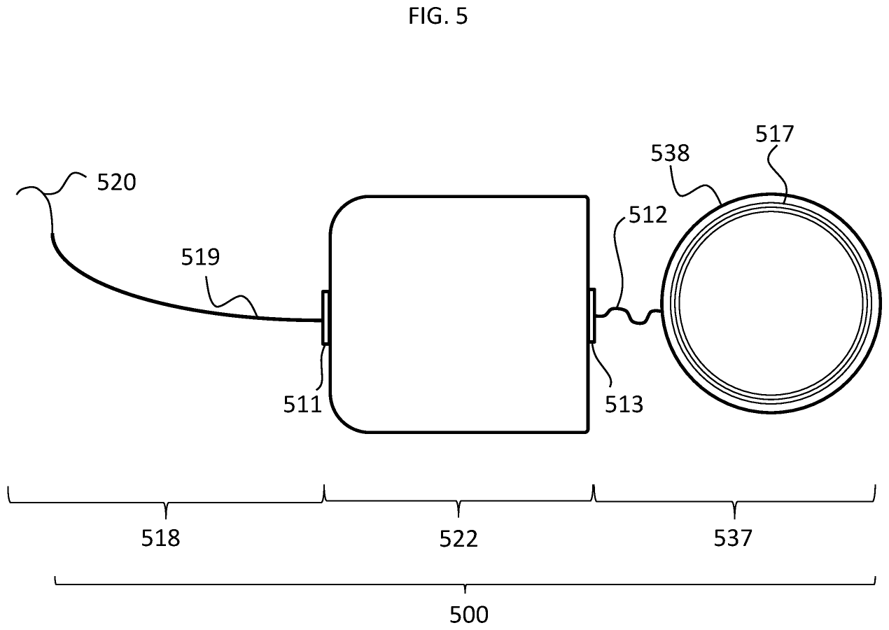

FIG. 5 depicts an exemplary alternate embodiment of an implantable component of a cochlear implant in a modularized form. Here, implantable component 500 corresponds to the implantable component 100 detailed above with respect to functionality and componentry, except that the electrode assembly is readily removable from the stimulator unit and the implantable coil is also readily removable from the stimulator unit (as opposed to the stimulator unit and the implantable coil being held together by the housing made of elastomeric material 199 as detailed above, and the elongate electrode assembly 118 being effectively permanently attached to the stimulator unit). More particularly, the implantable component 500 includes a receiver stimulator unit 522 that includes one or more feedthrough assemblies that permit signal communication with the coil 517 and the interior of the housing containing functional electronics of the cochlear implant, while maintaining hermetic sealing of that housing, and further includes one or more feedthroughs than enable communication with an electrode array to the receiver stimulator unit 522. In this regard, as can be seen, the implantable component 500 includes a coil unit 537 that includes a coil 517 located in a silicone body 538, and an electrical lead assembly 515 that is connected to a feedthrough 513 of the receiver stimulator unit 522, thus placing the coil 517 into signal communication with the electronic assembly of the receiver stimulator unit 522. On the opposite size of the stimulator unit 522 is feedthrough 511 of the receiver stimulator unit 522. Attached to the feedthrough 511 is the electrode assembly 518, which includes lead 519 to which is attached to electrode array 520 at the distal end thereof, the feedthrough 5111 placing the electrode array (via the lead 519) into signal communication with the interior of the receiver stimulator unit 522. In an exemplary embodiment, the connectors 510 and 512 are removable from the feedthroughs 511 and 513, respectively, thus enabling the electrode assembly 518 and the coil unit 537 to be removed from signal communication with the stimulator unit 522.

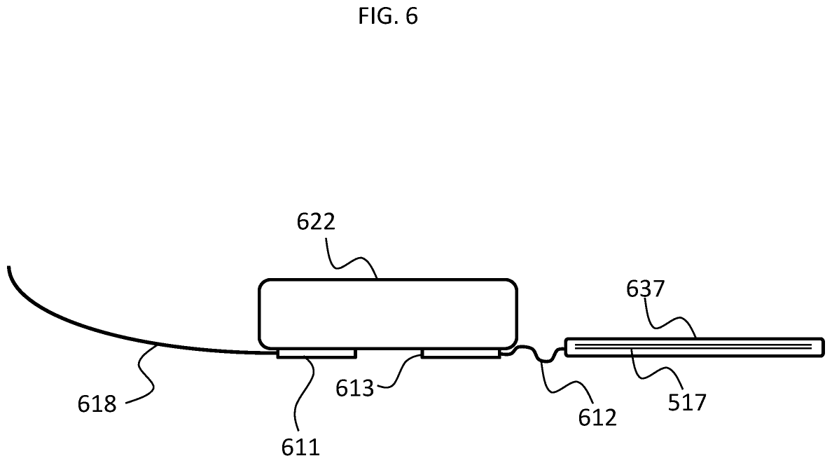

FIG. 6 depicts another exemplary alternate embodiment of an implantable component of a cochlear implant in a modularized form. As with implantable component 500, implantable component 600 corresponds to the implantable component 100 detailed above with respect to functionality and componentry. More particularly, the implantable component 600 includes a stimulator unit 622 that includes one or more feedthrough assemblies that permit removable attachment of the coil and the electrode array to the receiver stimulator unit 522. In this regard, as can be seen, the implantable component 600 includes a coil unit 637 that includes a coil 517 located in a silicone body, and an electrical lead assembly 612 that is connected to a feedthrough 613 of the receiver stimulator unit 622, thus placing the coil 617 into signal communication with the electronic assembly of the receiver stimulator unit 622. As can be seen, instead of the feedthrough 613 being on the side of the stimulator unit 622, it is on the bottom (the skull-facing side). Also, a feedthrough 611 of the receiver stimulator unit 622 is located adjacent feedthrough 613 on the bottom of the unit 622. Attached to the feedthrough 611 is the electrode assembly 618, which includes a lead to which is attached to electrode array at the distal end thereof, and includes connector 610 that is attached to feedthrough 611, thus placing the electrode array into signal communication with the stimulator unit 622.

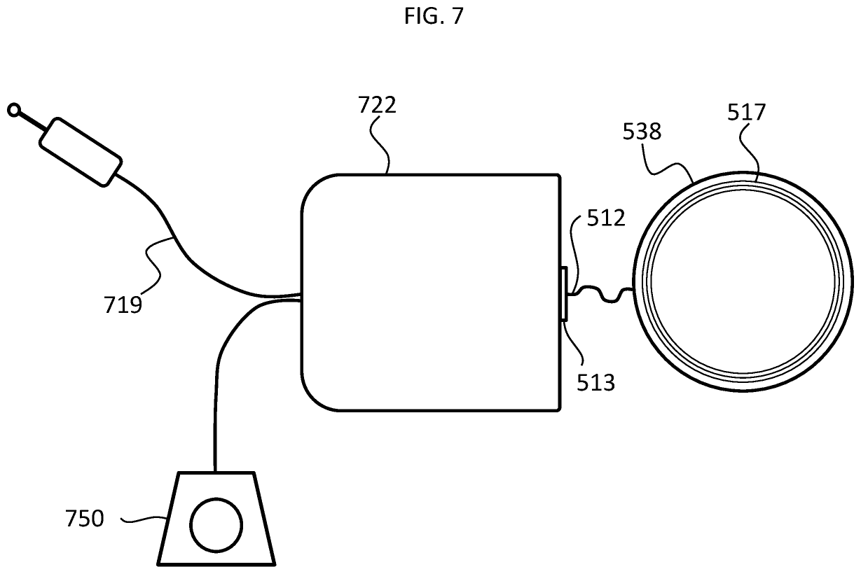

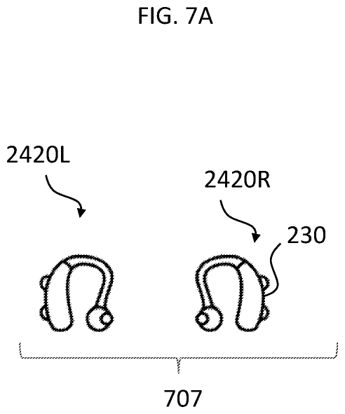

FIG. 7 depicts a totally implantable hearing prosthesis that includes a stimulating assembly 719 in the form of a DACS actuator (again, in keeping with the above, any disclosure of one type of output stimulating device corresponds to another disclosure of any other type of stimulation device herein, providing that the art enables such-thus, the disclosure of this DACS actuator corresponds to an alternate disclosure of a middle ear actuator or an active transcutaneous bone conduction device actuator, or a cochlear implant electrode array, or a retinal implant electrode array, etc., with the circuitry of the implant being different accordingly), and the hearing prosthesis further includes an implantable microphone 750. In this embodiment, the stimulating assembly and the implantable microphone are in signal communication with the electronics assembly located in receiver stimulator unit 722 via the same feedthrough or via separate respective feedthroughs. It is noted that the embodiment of FIG. 7 depicts a configuration where the feedthrough(s) are located on the bottom of the housing, and a feedthrough is also located on a side of the housing. It is noted that in some embodiments, all of the feedthroughs are located on the bottom of the housing. The embodiment of FIG. 7 is presented to show that the various configurations of feedthrough locations can be combined in some embodiments.

In view of the above, it is to be understood that in an exemplary embodiment, there is a device is hermetically sealed and is implantable, which includes a housing. The housing contains circuitry of a hearing prosthesis, and corresponds to the housing detailed above or variations thereof having opening(s) in which feedthrough assembly(ies) are located in the opening(s). The housing can also contain a battery so that the device can be “self powered” and thus be a totally implantable hearing prosthesis.

Embodiments include a modified version of the implantable component 100 as detailed above, and will be described below, but first, some background information on external components.

FIG. 7A shows another exemplary embodiment of a hearing prosthesis system 707 in the form of a left side and right side conventional hearing aid system. Element 2420L is a left-side hearing aid, and 2420R is a right side hearing aid, which would be worn on the left ear and the right ear, respectively, of a recipient. The two BTE devices can be utilized in a bilateral arrangement (conceptually shown in FIG. 7A—there would be a human head in between the two devices and the BTE devices would extend from a front of the respective pinnas to behind the respective pinnas in a traditional manner). Embodiments can include one or more of the features of BTE device 242 detailed above, and will not be repeated in the interests of textual economy.

FIG. 2C presents additional details of an external component assembly 242, corresponding to external component 142 above.

External assembly 242 typically comprises a sound transducer 291 for detecting sound, and for generating an electrical audio signal, typically an analog audio signal. In this illustrative arrangement, sound transducer 291 is a microphone. In alternative arrangements, sound transducer 291 can be any device now or later developed that can detect sound and generate electrical signals representative of such sound. An exemplary alternate location of sound transducer 291 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 216 (sometimes referred to as a headpiece) comprises an external coil 228 (which can correspond to coil 130 of the external component of FIG. 1A) and, a magnet (not shown) secured directly or indirectly to the external coil 228. The signal processing unit processes the output of microphone 291 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals-because element 230 contains this circuitry, the entire component 230 is often called a sound processing unit or a signal processing unit. These coded signals can be referred to herein as a stimulation data signals, which are provided to external transmitter unit 296 via a cable 247. In this exemplary arrangement of FIG. 2C, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 2C that utilizes the bayonet connector.) Conversely, in some embodiments, there is no cable 247. Instead, there is a wireless transmitter and/or transceiver in the housing of component 230 and/or attached to the housing (e.g., a transmitter/transceiver can be attached to the receptacle 219) and the headpiece (transmitter unit 296) can include a receiver and/or transceiver, and can be in signal communication with the transmitter/transceiver of/associated with element 230.

FIG. 1E provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1E, an ITE component 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). ITE component 250 includes a housing 256, which can be a molding shaped to the recipient. Inside ITE component 250 there is provided a sound transducer 291 that can be located on element 250 so that the natural wonders of the human ear can be utilized to funnel sound in a more natural manner to the sound transducer of the external component. In an exemplary arrangement, sound transducer 242 is in signal communication with the remainder of the BTE unit via cable 252, as is schematically depicted in FIG. 1E via the sub cable extending from sound transducer 242 to cable 252. Shown in dashed lines are leads 21324 that extend from transducer 291 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal.

It is noted that in at least some exemplary embodiments, there is no in the ear component 250 and thus no lead 252. In this regard, the arrangement of FIG. 2C is part of a bimodal hearing prostheses, that includes a conventional acoustic hearing aid functionality, and also implantable stimulation such as by way of the above example a cochlear implant, although in other embodiments, such could be a middle ear implants or bone conduction device or some other arrangement.

Also, FIG. 2C shows a removable power component 270 (sometimes battery back, or battery for short) directly attached to the base of the body/spine 230 of the BTE device. As seen, the BTE device in some embodiments include control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

The above description presents baseline technologies that are not innovative and do not form the basis of the invention herein. In at least some exemplary embodiments, the teachings above are used in combination with the innovative teachings below. Further, in at least some exemplary embodiments, the teachings above are modified so as to implement the innovative teachings below. In this regard, in at least some exemplary embodiments, the above is modified so as to enable the use thereof with the teachings herein. However, any embodiment below can utilize one or more of the teachings above in combination and/or by modification.

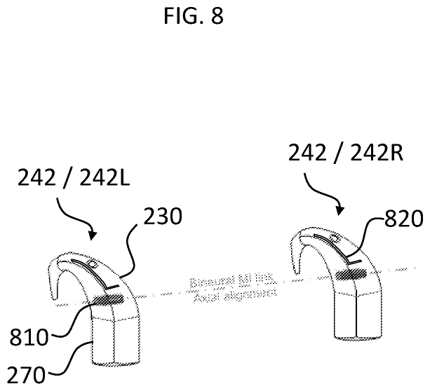

FIG. 8 presents some additional features of the exemplary external system 242, along with an exemplary arrangement of use in a bilateral hearing prosthesis system. Here, as can be seen, there is a left external assembly 242L and a right external assembly 242R. In this exemplary embodiment, the external assemblies correspond to those of FIG. 2C (some portions of the assemblies are not shown, such as the headpiece (transmitter unit) and the ITE component-it is noted that in some embodiments these components are optional and may not be present, and thus the arrangement of FIG. 8 can depict the outer profile of these devices somewhat accurately), but can also correspond to those of FIG. 7A, etc. (various components of one arrangement can be used in another, so in the interest of textual economy, we disclose that any teaching herein can be combined with one or more other teachings herein unless otherwise noted, provided that the art enables such).

In this exemplary embodiment, as can be seen, the external assemblies 242 include cylindrical antennas (sometimes called rod antennas) 810. These are generally arrayed within the spine of the BTE device such that when utilized in the bilateral arrangement (conceptually shown in FIG. 8—there would be a human head in between the two devices and the BTE devices would extend from a front of the respective pinnas to behind the respective pinnas in a traditional manner), the axis about which the respective coils of the antennas are wound would lie on the same axis as shown/would be at least generally aligned. In an exemplary embodiment, the MI radio antennas are utilized to communicate between the two external components in a bilateral arrangement. Embodiments include MI radio antennas that are utilized to both communicate between the external components and the implanted components. In an exemplary embodiment, the antennas and the systems associated there with can be one way (send or receive) or can be two-way (send and receive). It is briefly noted that the concept of FIG. 8 would also be applicable, in at least some exemplary embodiments, to utilization of MI-radio in a bilateral system that utilizes in-the-ear devices, such as a totally in the ear device or an in-the-ear device where the MI radio antennas are located in the ear canal approximate thereto or otherwise on the side of the pinna opposite that which results when the behind the ear device arrangement is utilized of FIG. 8.

FIG. 8 also shows a Bluetooth antenna 820 located on the spine 230. In an embodiment, theses antennas are part of or are connected to a Bluetooth chip. Thus, embodiments include communication arrangements at the 2.4 GHz area and ranges thereabout. Other regimes of communication can be used in some embodiments. Also, it is noted that some embodiments include only one component that has a Bluetooth operating system, or at least a full operating system. Only one component may have a Bluetooth antenna. Thus, some embodiments include an arrangement where of the two components of the supplemental sensory system, only one component has a Bluetooth chip. There are embodiments where portions of a Bluetooth protocol or communication protocol are located and/or only run on one of the two components. Any layer of a protocol can be limited to one of the two components and/or excluded from one of the two components unless otherwise noted providing that the art enables such. More on this below.

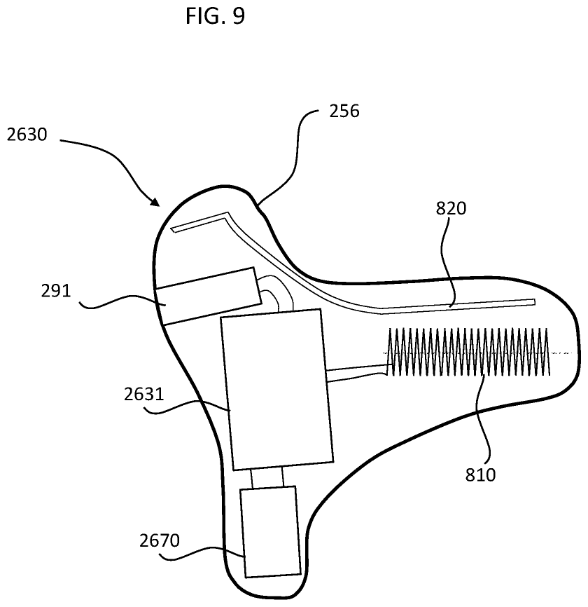

FIG. 9 presents another exemplary embodiment of an in-the-ear device 2630 having utilitarian value with respect to the teachings herein. This device is a fully contained external component of a cochlear implant or a middle ear implant or a DACS or an active transcutaneous bone conduction device or a conventional hearing aid (receiver not shown). In this exemplary embodiment, a microphone 291 is supported by housing 256 which is in signal communication via leads to a sound processor 2631. In an exemplary embodiment, the sound processor 2631 can be a miniaturized version of the sound processor utilized with the embodiments detailed above, and can be a commercially available sound processor that is configured for utilization within an ITE device. As seen, there is a battery 2670 that provides power to the system. Consistent with the teachings above, there is a cylindrical antenna 810 that is in signal communication via leads with the sound processor 2630. In this exemplary embodiment, the ITE device 2630 communicates with the implanted component via MI radio in a manner concomitant with the teachings detailed herein with respect to the ITE device that is in signal communication with a BTE device. Also included in the ITE device is a Bluetooth antenna 820 and associated circuitry (the antenna 820 can be part of a Bluetooth chip in an embodiment). Again, some components may not have the Bluetooth system, or not the full system.

It is also noted that some exemplary embodiments include an MI radio antenna and a Bluetooth antenna located in an OTE (off the ear) device. In an exemplary embodiment of this arrangement, this is a device that is located and otherwise magnetically held over the implanted wide diameter coil 137 of the implant, and does not have a component that is in contact with the pinna that is physically connected to the OTE device. There could be such a device that is in radio signal communication there with, and there could be an ITE device that is in radio signal communication therewith, but there is no physical link between the two—the link is electromagnetic. To be clear, any disclosure herein with respect to functionality and/or structure of a BTE device corresponds to an alternate disclosure of such with respect to an ITE device and an OTE device and vice versa two more times, unless otherwise noted and unless the art does not enable such.

Antenna 810 can be part of a magnetic inductance radio (MI radio) system that enables the establishment of a utilitarian ipsilateral communication link between the external component and the implant device. The communication link may operate between 148.5 kHz and 30 MHz by way of example only and not by way of limitation (the link between the coil 137 and coil 130 can be, in some embodiments by way of example only and not by way of limitation, less than 30 MHz, such as between 3 and 15 MHz in general, and more specifically, 4.5 MHz and 7 MHz).

It is noted that the teachings herein, while generally described in terms of transcutaneous communication, are also applicable to subcutaneous communication. That is, embodiments can be applicable to communication between two different antennas that are both implanted within a recipient. This can be, for example, where there is utilitarian value with respect to maintaining a hermetic body, such as a housing, without the risk of utilizing a feedthrough or the like therethrough. By way of example only and not by way of limitation, an antenna within a ceramic housing also containing a processor can communicate with a separate component that includes an implanted microphone. The utilization of the antenna in the housing can avoid the need for a feedthrough or the like from the component with the implanted microphone. Accordingly, any disclosure herein relating to transcutaneous communication also corresponds to a disclosure of subcutaneous communication unless otherwise noted providing that the art enables such.

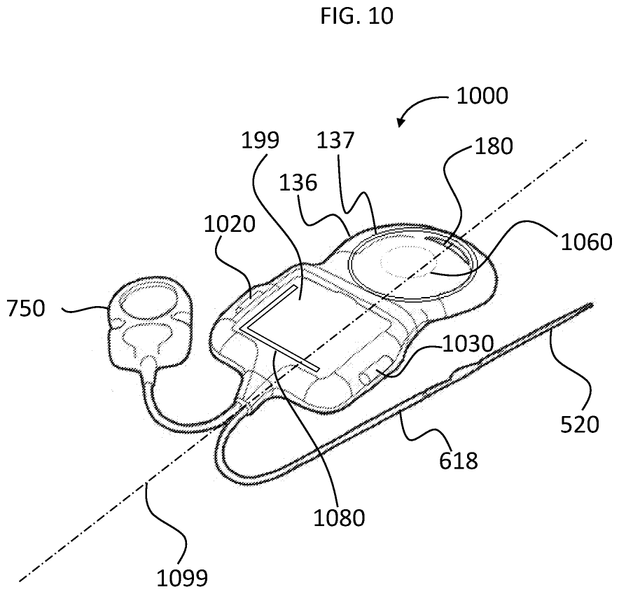

With the above as background, embodiments of some teachings are such that the physical implementation of the MI-radio antennas of the implant for ipsilateral communication with the external component are well-defined as such to provide, and in some instances, guarantee, strong incoming MI implant signals. Accordingly, in an exemplary embodiment, as seen in FIG. 10 there is an implantable component 1000, with some of the reference numbers reused to demonstrate like components. FIG. 10 depicts an isometric view of the component 1000, along with the longitudinal axis 1099 of the receiver-stimulator for future reference. For example, there is the silicone overmold 136 and 199, overmolding the coil number 137 and the housing of the receiver stimulator (the housing is not seen in FIG. 10—it is under the silicone). The implant includes cylindrical coil antenna 1020 (where antenna 1030 is located on the opposite side of the implant, and eclipsed by a portion of the housing. Also shown is Bluetooth antenna 1080, where the implant includes circuitry to support Bluetooth communication.

Embodiments include utilizing wireless signals (electromagnetic signals, signals in the megahertz range, signals in the gigahertz range (1 to 10 GHz), etc.) to provide/ascertain/develop an estimation of relative direction and/or distance and/or location of a device that is outputting, such as streaming data, relative to a component of a sensory prosthesis, such as by way of example, a right side conventional behind the ear device hearing aid. More specifically, embodiments use radio signals, such as the 2.4 GHz frequency signals of Bluetooth Low Energy protocols, that can provide an estimation of relative direction and/or distance and/or a vector path between two devices. This estimation can rely on any one or more algorithms and measurements, such as angle of arrival (AOA) or angle of departure (AOD) algorithms, RSSI (Received Signal Strength Indicator), trilaterion, triangulation. Bluetooth direction finding can be used. Any device, system, and/or method that can enable the teachings detailed herein vis-à-vis direction, distance and/or location, or any spatial regime having utilitarian value of one element relative to another element or one element of a global can be utilized in at least some embodiments providing that such has utilitarian value.

Embodiments herein focus on the utilization of two or more components of a system. While embodiments often focus on the utilization of Bluetooth, it is noted that any other protocol than Bluetooth can be utilized providing that there is utilitarian value according to the teachings detailed herein providing that the art enable such. For example, as detailed above, MI radio links can be utilized. Note also that embodiments include utilizing different protocols for different components. For example, one component, such as the external component or the implanted component, can utilize the Bluetooth protocol, and the other component can utilize MI radiolink protocol (a third protocol can exist to communicate between the two devices, such as a traditional transcutaneous inductance communication protocol, where via back telemetry, the implant can communicate with the external device (the external component can communicate with the implant by the traditional transcutaneous communication)—note that the components can use MI radio or Bluetooth to communicate with each other in some embodiments—in some embodiments, both can have MI radio but only one has Bluetooth for example). Neither the external nor the implanted component could use Bluetooth for that matter. Both could use MI radio or two different protocols none of which include Bluetooth (or MI radio for that matter).

In this regard, a Bluetooth chip may not be in both components. Embodiments include one or both components that do not have a Bluetooth chip or otherwise do not have a Bluetooth protocol. Embodiments include one component having a Bluetooth chip and one that does not have such and/or does not have a Bluetooth protocol.

There can be utilitarian value with respect to beamforming an outputted radio wave between the transmitter and receiver. In an exemplary embodiment, this can minimize the spatial power the transmitter transmits in directions that are not in line with the receiver, reducing the power usage of the transmitter and/or reducing collisions in crowded areas, the latter having much utilitarian value with respect to a classroom setting or a theater setting, etc., by way of example only and not by way of limitation. Such can also have utilitarian value with respect to choosing which transmitter to use to transmit data to a given sensory device, such as a conventional hearing aid, if multiple transmitting devices and/or multiple antennas are present in an environment of the conventional hearing aid (by example).

Embodiments include utilizing two components of a sensory supplement system, such as a left side external component of a hearing prosthesis system and a right side external component of a hearing prosthesis system, or an external component and an implanted component of a hearing prosthesis system, one of which or both of which have some form of Bluetooth capability for example, or any equivalent technology, to implement some exemplary teachings herein. In an exemplary embodiment, there is a division of “labor” between the two components (labor associated with communication/a division of communication load). One component (e.g., a left conventional BTE hearing aid (which is an external component irrespective of whether there is an implanted component, which there would not be with a conventional hearing aid barring a bimodal system)) is utilized to receive and process data that is transmitted to the sensory supplement system, such as by a data stream, and one component (e.g., a right conventional BTE hearing aid) is utilized to execute the spatial functionality features of the teachings detailed herein. Also, concomitant with the teachings above, in an embodiment, one of the components can be an implanted device, and another component can be the external device (e.g., the device that provides power to the implant, such as the external component of a cochlear implant, or a separate acoustic hearing aid) or another external device (such as a hand-held “assistant” device—more on this below).

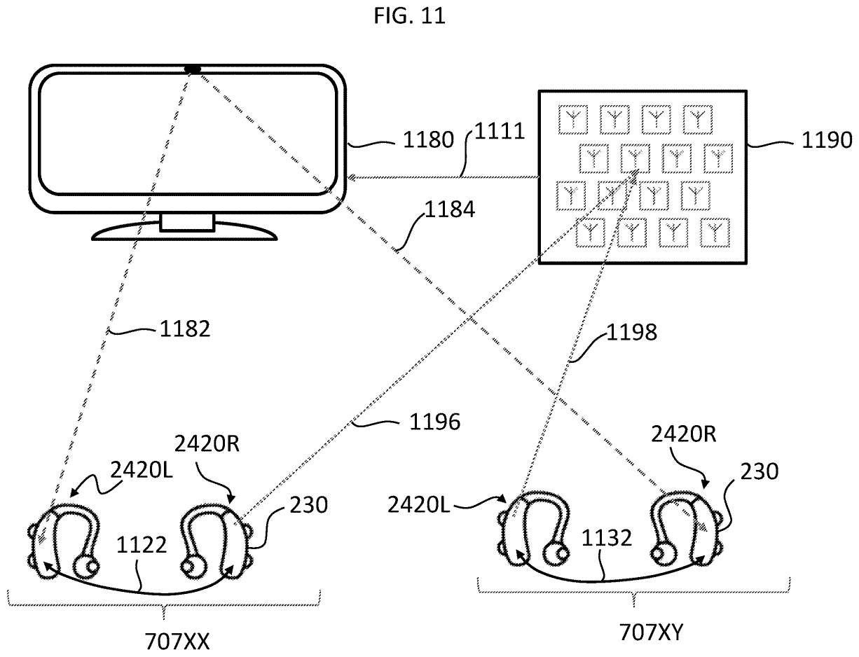

FIG. 11 presents an exemplary scenario where there are two sensory supplement systems 707XX and 707XY, worn by respective people (not shown), both receiving streaming audio from television 1180. The audio stream from television 1180 is represented by dashed arrow 1182 and dashed arrow 1184, which correspond to Bluetooth standard transmissions, where the respective audio streams are received by the left hearing aid of the system on the left and the right hearing aid of the system on the right (again, received using the Bluetooth standard, where the hearing aid is Bluetooth compatible). Meanwhile, the right-side hearing aid of system 707XX outputs direction finding data (again, using the Bluetooth standard in an exemplary embodiment, full or partial) to the antenna array 1190, while the left side hearing aid of system 707XY outputs direction finding data to the antenna array 1190, the direction finding data represented by arrows 1196 and 1198 respectively. Antenna array 1190 communicates data based on the received direction finding data signals to streaming device 1180 or a device that controls at least some aspects of streaming device 1180 via link 1111 (which can be wired or wireless) and based on that data that is received by streaming device 1180 or the controller thereof, streaming device 1180 directs the audio stream(s) in a direction (e.g., via. Beamforming) and/or at a certain power. The direction would be directed to the pertinent hearing aids (which may or may not have an offset owing to the fact that the directionality is based on the hearing aid that is not receiving the streamed data-the offset between the two hearing aids will not impact performance in some embodiments, and thus it can be sufficient to have the signal directed to the hearing aid executing the spatiality functionality), and the power, in an exemplary embodiment, is based on the distance of the hearing aids from the streaming device 1180. Thus, in an embodiment, there is an audio source that modifies its output, such as a Bluetooth stereo audio stream, based on which hearing aid the audio source is streaming to and/or their relative position, and/or global position, obtained using, for example, Bluetooth direction finding in conjunction with the antenna array.

In this regard, Bluetooth direction finding can be present in one or both of the components of the system. Indeed, as noted herein, the work split can shift between components depends on needs or for arbitrary reasons. And note that embodiments may not include Bluetooth direction finding. One or both components could be completely devoid of such. Any other spatiality function regime that can have utilitarian value can be utilized in some embodiments. That said, in some embodiments, one or both components include both Bluetooth direction finding and another directionality finding regime. Any direction finding regime or combinations thereof they can have utilitarian value can utilize at least some exemplary embodiments.

Note that while embodiment are often described in terms of audio streaming, embodiment can include video streaming, which has utilitarian value with respect to a retinal implant. For example, the external device of a retinal implant can process the streaming, and the implant can execute the directionality/spatial functionality, or visa-versa.

It is briefly noted that in this exemplary embodiment, consistent with the “labor” sharing concepts presented above, the left hearing aid of system 707XX processes the audio stream 1182, which is received via the Bluetooth system of the left hearing aid, and then transmits a signal to the right hearing aid of system 707XX such as via the use of the MI radio system thereof as represented by link 1122 (in an embodiment, this is not a Bluetooth link, while in other embodiments, it can be a Bluetooth link, any wireless system of transmission that will enable the teachings herein can be used, and in some embodiments, the link is a wired link). The same can also be the case with respect to the hearing aids of system 707XY vis-à-vis link 1132. The transmitted audio signal by the MI radio systems require less processing power, in some embodiments no processing power, to convert into output and/or to manipulate by the receiving component into source data upon which to evoke a hearing percept, as contrasted to the audio stream received over signal paths 1182 and 1184. In this exemplary embodiment, the communication links 1122 and 1132 are unidirectional, while in other embodiments, they can be bidirectional. Again, while MI radio has been described above as establishing the links 1122 and 1132, in other embodiments, other types of communication regimes can be utilized to communicate the processed data from one component to the other component. In an embodiment, a mono audio stream is outputted from the hearing aid that received and processed the streamed data to the hearing aid responsible for spatial functionality. Also, the left and/or right hearing aids can be configured for information data exchange between them (e.g., location information can be sent over the links 1122 and 1132 (if they are two way links) or another link to the hearing aid that is processing the audio source).

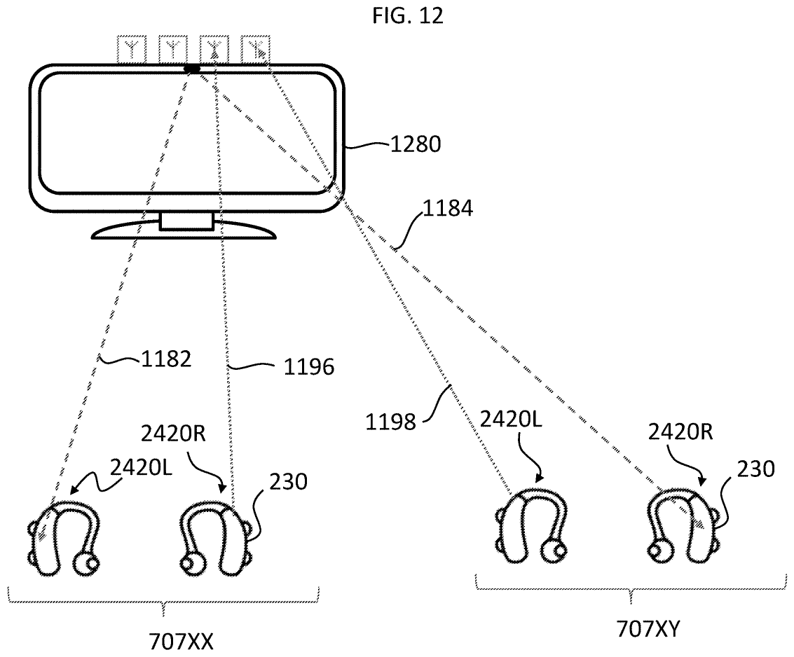

FIG. 12 presents an alternate exemplary embodiment except where the antenna arrays combined with the streaming device 1280, as contrasted to the arrangement of FIG. 11, where there are two separate “infrastructure” components-the array and the streaming device.

Embodiments include variable division of labor between the various components of the sensory supplement devices. In an embodiment, the sensory supplement systems are configured to “self-determine” what component should do what function. In an exemplary embodiment, the decision as to the division of labor can be arbitrary or can be based on various factors. For example, in the scenario depicted in FIG. 11, system 707XX is assigned the task of receiving and processing the audio data because the battery charge level of the left hearing aid was higher than that of the right, and the opposite is the case with respect to system 707XY. The systems can be configured to take into account other factors that can come into play with respect to making that decision, such as whether or not there is a head shadow effect with respect to one or the other devices, the degree to such, etc.

In an embodiment, the left hearing aid is hard designed to receive and process the data stream, while the right hearing aid is hard designed to handle the spatial functionality. In this embodiment, the left hearing aid always processes and receives the data stream, and the right hearing aid cannot do such, and the right hearing aid always executes the function related to spatiality, and the left cannot do such. In an embodiment, it could be that only the left hearing aid can transmit the signal based on the processed data and the right hearing aid can only receive that transmitted signal (e.g., via the MI radio link). The right hearing aid cannot transmit a signal to the left hearing aid, and the left hearing aid cannot receive that signal even if the right hearing aid transmitted such. All of this said, in an alternative embodiment, it can be a software and/or firmware implementation that controls which hearing aid functions accordingly, and thus instead of being hard designed to function accordingly, they are soft designed to function accordingly. Still, in an embodiment, a control switch or the like can be utilized to control functionality. That is, a user can select which component will do what. We note further that in an embodiment, the remote assistant to be utilized to control which component does what. Still, embodiments can include “smart” systems that can evaluate a state of one or both components and divide the labor accordingly, based on variable factors, such as battery power level, head shadow, etc.

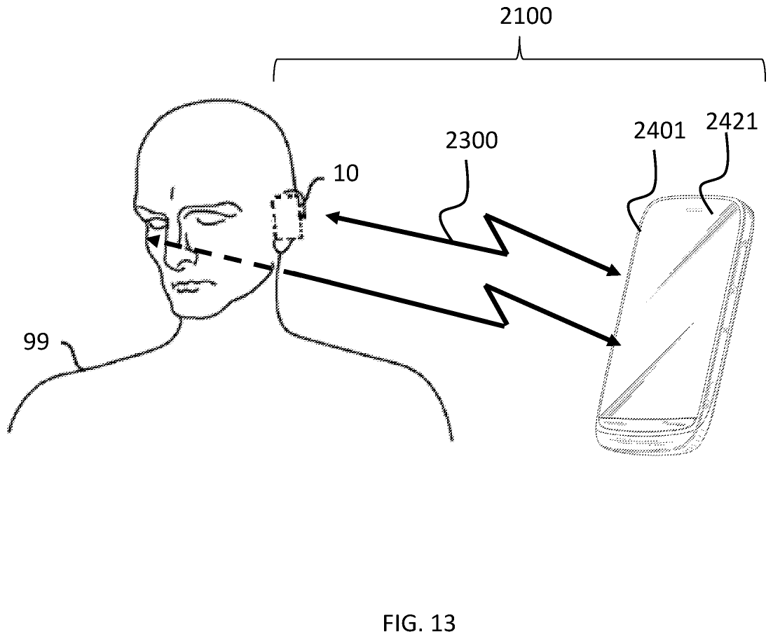

With reference to an assistant, FIG. 13 depicts an exemplary system 2100 according to an exemplary embodiment, including hearing prosthesis system 10, which, in an exemplary embodiment, corresponds to cochlear implant system 10 detailed above, and a portable body carried device (e.g., a portable handheld device as seen in 13, a watch, a pocket device, etc.) 2401 in the form of a mobile computer having a display 2421. Device 2401 is an assistant device (more on this in a moment). The system includes a wireless link 2300 between the portable handheld device 2401 and the hearing prosthesis 10. In an embodiment, the prosthesis 10 is an implant implanted in recipient 99 (represented functionally by the dashed lines of box 10 in FIG. 13). Not seen in FIG. 13 is the second right side cochlear implant system. Link 2300 is also in communication with that cochlear implant system. Thus, the assistant 2401 can communicate with two or more components of a sensory supplement device.

In an exemplary embodiment, the system 2100 is configured such that the hearing prostheses 10 and the portable handheld device 2401 have a symbiotic relationship. In an exemplary embodiment, the symbiotic relationship is the ability to display data relating to, and, in at least some instances, the ability to control, one or more functionalities of the hearing prostheses 10. In an exemplary embodiment, this can be achieved via the ability of the handheld device 2401 to receive data from and/or provide instructions to the hearing prosthesis 10 via the wireless link 2300 (although in other exemplary embodiments, other types of links, such as by way of example, a wired link, can be utilized). This can be achieved via a Bluetooth link (link 2300 can be a Bluetooth link) or by some other communication arrangement. This can be achieved via communication with a geographically remote device in communication with the hearing prosthesis 10 and/or the portable handheld device 2401 via link, such as by way of example only and not by way of limitation, an Internet connection or a cell phone connection. In some such exemplary embodiments, the system 2100 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 10. Some of these embodiments will be described below).)

In an exemplary embodiment, the portable handheld device 2401 is configured to receive data from a hearing prosthesis and present an interface display on the display from among a plurality of different interface displays based on the received data. The portable handheld device 2401 can be configured to provide instructions to the hearing prostheses. In an exemplary scenario, the portable handheld device 2401 can receive data such as battery level, signal strength, etc., from one or both components of the sensory supplemental system, and evaluate that receive data, and assign labor tasks to the different components. If the portable handheld device determines that the signal strength of one component is or will be stronger or otherwise superior to that of the other component, the portable handheld device 2401 will assign the task of receiving and processing the stream data to that one component. The portable handheld device will also assign the spatiality functionality to the other component. The portable handheld device 2401 can be configured to continuously or periodically monitor one or more of the features associated with the various components and can make a determination to swap or change the divisional labor based on updated data.

Note that in alternate embodiments, the portable handheld device 2401 is not necessary to implement the teachings detailed herein. In an embodiment, one or both of the components can evaluate the data and divide the division of labor accordingly. To ensure that there is no endless do loop, one component can be provided as the default master. This decision can be arbitrary. But to be clear, any functionality detailed herein with respect to the division of labor associated with the portable handheld device to go to for a one can be executed by one or both of the components of the sensory supplement system that are worn on the body and/or implanted in the body and such devices can be configured to do so unless otherwise noted, providing that the art enables such. Note further that in an exemplary embodiment, the portable handheld device 2401 can execute the spatiality functionality for example and/or can receive the streaming data and process such for example. In this embodiment, only one component of the prosthesis system that is worn or implanted would execute the other functionality.

In view of the above, there is a system, such as a bilateral conventional hearing aids system or a bilateral cochlear implant system or a unilateral cochlear implant system, unilateral only having an external component and an internal component on one side of the recipient. In an embodiment, the system comprises a first device and a second device. In this embodiment, the first device is a component of a sensory prosthesis configured to receive a data stream and evoke a sensory percept based on the data stream. In an embodiment, this can be the left-hand side or right-hand side conventional hearing aid. This could be the external component of a cochlear implant, or could be the implanted component of cochlear implant. This can be any of the components detailed herein that are part of a sensory supplement system as detailed herein.

In this embodiment, the second device is configured to provide spatial output to the first device and/or another device remote from the second device, where the another device could be the component in the infrastructure, such as television 1280.

Spatial output, including localization output can be any signal that can be utilized to spatially reference the second device or any other device applicable to the teachings detailed herein. In an exemplary embodiment, this can be a signal output by the second device's Bluetooth system, where one of the environmental components (e.g., television 1280, or the array noted above in FIG. 11) uses a signal for angle of attack purposes to determine the direction of the second device. Spatial output can be output usable to determine a direction (e.g., a simple direction, such as 10 degrees to the right, 30 to 35 degrees to the left) or a vector (30 degrees to the left, 5 degrees up elevation). This can also be a more sophisticated output, which could be for example two-dimensional or three-dimensional Cartesian coordinate values with reference to a frame of reference. This could be global positioning system information. Still, in an embodiment, this can be a transmission over the Bluetooth antenna of the second device that can be received by the array of the device in the environment where the infrastructure device can develop spatial data therefrom.

Note also that it could be that the second device is configured with a receiver or transceiver that is configured to receive an output from the antenna array of the component in the environment and is configured to utilize angle of departure (the signal could contain angle of departure information, which signal is received by the second device) and/or angle of arrival techniques to ascertain the direction of the transmitter. The second device can then convey spatial output based on this ascertained directionality to the first device or to another device remote from the second device, the another device could be the component in the environment. In a method involving utilizing the system, the component in the environment could then execute a beamforming operation for example and direct the data stream to the first device (or the second device, where that would be close enough for utilitarian receipt by the first device).

It is noted that the phase of the outputted signal by the second device could also be utilized to implement the locationality functions taught herein. Also, Bluetooth direction finding signals can be utilized. In this regard, the outputted signal by the second device could be a Bluetooth direction finding signal.

It is also noted briefly that while embodiments herein are sometimes directed towards generally positionally static elements of a given system, embodiments also include scenarios where one or more elements of the system are dynamic and otherwise moving. For example, in an embodiment a recipient may be walking or running or otherwise moving within an environment where there is streaming data or otherwise where there is an environmental component. Embodiments include tracking the location of the recipient, or more accurately, tracking the position of the one or more components involved in the spatiality methods detailed herein, or tracking can include two or three dimensional positioning or otherwise directionality or vector determination.

This second device could be the right-hand side of the conventional acoustic hearing aid (where the first device is the left-hand side). This could be the portable handheld device 2401 noted above. Where the first device is the external component or the internal component of an implantable medical device, such as a retinal prosthesis or a middle-ear implant or a cochlear implant or an active transcutaneous bone conduction system for example, the second device can be the other of the external component or the internal component.

In an embodiment, the first device is a hearing prosthesis component (e.g., left or right side conventional acoustic hearing aid, implanted cochlear implant component, external component of an active transcutaneous bone conduction device, etc.), and the data stream is an audio stream (streamed over a Bluetooth signal from a component in the environment, for example, such as a television, a computer (desktop or laptop), or a Bluetooth music radio, or some other component, or an automobile Bluetooth, etc.). In an embodiment the second device is configured to provide the spatial data, such as spatial data to the first device and the first device is configured to control a directionality feature of a receiver and/or transceiver based on the spatial data. In an embodiment, this could be a receiver/transceiver of the first device. In this regard, embodiments can include a receiver/transceiver that has a reception directionality feature so that it focuses reception in a certain direction to the exclusion of other directions. In an embodiment, the receiver ignores signals that come from directions other than the direction of interest. In an embodiment, the receiver provides weighting functions to the signals, so that signals coming from certain directions will be amplified more than signals that come from other directions. Indeed, in an embodiment, only signals coming from a certain direction will be amplified.

In an embodiment, the system includes the another device (e.g., television 1280, array 1190), etc. In an embodiment, the second device is configured to provide the spatial output to the another device (e.g., over a Bluetooth link). In this embodiment, the another device is configured to control and/or provide data for control of a directionality feature of a transmitter that transmits the data stream based on the spatial output so that the data stream is directed more towards the second device than that which would have been the case in the absence of the provided spatial output. In an exemplary embodiment, the array 1190 can directly control the beamforming features of the television 1180 or signal 1111 can be used by television 1182 as a basis for beamforming to the sensory supplement system. Thus, in an exemplary embodiment, the another device can include the transmitter and/or transceiver and in an exemplary embodiment, the transmitter and/or transceiver is part of a device separate from the another device.

In an embodiment, the first device is an external component of the sensory prosthesis, wherein the first device is configured to transcutaneously communicate with an implantable component of the sensory device, and the second device is an external component of a second sensory prosthesis, wherein the second device is configured to transcutaneous communicate with an implantable component of the second sensory device. This can be a so-called bilateral cochlear implant, where there are implants in both cochleas and thus two external devices. Accordingly, in an embodiment of this embodiment, the sensory prosthesis and the second sensory prosthesis are a same type of sensory prosthesis. That said, in an embodiment, the sensory prosthesis and the second sensory prostheses are different types of sensory prostheses. This can be for example a so-called bimodal arrangement, where for example there is a conventional acoustic hearing aid on the left side, and a cochlear implant on the right side, or vice versa. Note also that there could be a bone conduction device on one side and a cochlear implant on the other or any other combination. Note further that embodiments are not necessarily limited to different types of devices on one side. The aforementioned bimodal arrangement can be located on the same side of the recipient. In this regard, say that the right side cochlea of a recipient no longer outputs electrical signals for medium and high frequencies. However, the cochlea will output electrical signals for low frequencies. A so-called short electrode array could be located in the cochlea, and a cochlear implant can be utilized to provide hearing at medium and high frequencies. Also, the right side of the recipient can also have a conventional acoustic hearing aid to amplify low-frequency signals. This would be sensory prostheses that are of different types but located on the same side of the head.

Still, with reference to the embodiment of FIG. 11, in an exemplary embodiment, the first device can be a conventional acoustic hearing aid, and the second device can be a second conventional acoustic hearing aid.

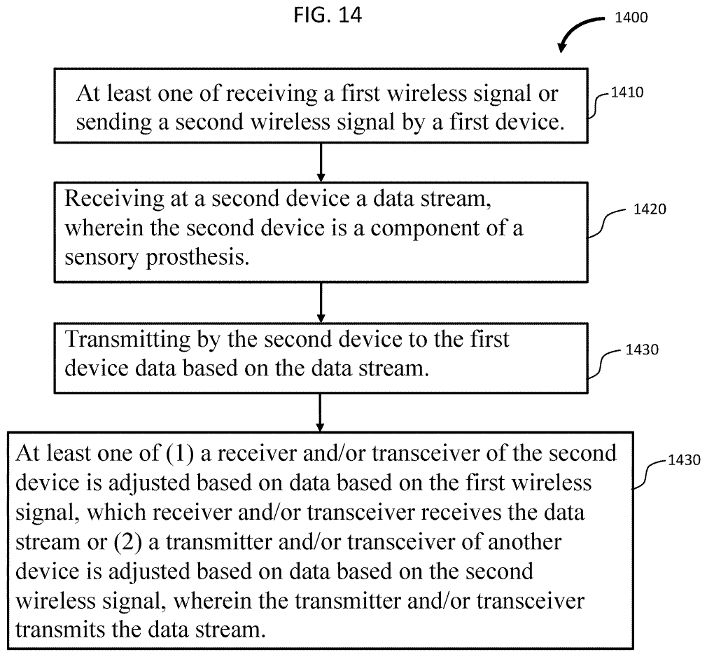

Embodiments include methods. FIG. 14 presents an exemplary flowchart for an exemplary method, method 1400, which includes method action 1410, which includes the action of at least one of receiving a first wireless signal by or sending a first signal from a first device. The first wireless signal can be the signal from the antenna array 1190 or the television 1280. The second wireless signal can be the signal output by any of the components detailed above. By way of example, the second wireless signal can be signal 1196 from hearing aid 2420R.

Method 1400 further includes method action 1420, which includes the action of receiving at a second device a data stream, wherein the second device is a component of a sensory prosthesis. In this regard, the second device can be hearing aid 2420L, and the data stream can be the datastream 1182. Note that in this method, the first device can be, but need not be, a component of a sensory prosthesis. The first device could be the assistant 2401 of the prostheses system 2100 by way of example only.

Method 1400 further includes method action 1430, which includes the action of transmitting by the second device to the first device data based on the data stream. This can be data transmitted by the MI radio signal over link 1122 for example. In this method, in an exemplary embodiment, at least one of (1) a receiver and/or transceiver of the second device is adjusted based on data based on the first wireless signal, which receiver and/or transceiver receives the data stream or (2) a transmitter and/or transceiver of another device is adjusted based on data based on the second wireless signal, wherein the transmitter and/or transceiver transmits the data stream. With respect to the receiver and/or transceiver being adjusted, here, this could be the tuning of the Bluetooth system of the second device to focus on the signal from the environmental components, such as television 1180. With respect to the transmitter and/or transceiver of the another device, this could be the beamforming of the output of the television noted above. These actions can be executed based on the teachings above.

Consistent with the teachings above with respect to labor splitting, in an exemplary embodiment, the first device does not receive the data stream. Granted, the signal from the environmental component may and likely will impinge upon the Bluetooth system antenna of the second device. However, this signal will not be used by the second device and otherwise will not be processed by the second device. Thus, it will not be received. Further, in an exemplary embodiment, the method comprises receiving by the first device the data based on the data stream, wherein the first device evokes a sensory prosthesis based on the received data based on the data stream. In this regard, with reference to the conventional acoustic hearing aid system detailed above, the second device will process the data stream and utilize the datastream to evoke a hearing percept in the pertinent ear. For example, if the second device is the left acoustic hearing aid, that acoustic hearing aid will process the stream signal and will provide an electrical signal to a receiver (speaker) of that hearing aid to evoke a hearing percept in the left ear based on that stream signal. If the first device is the right-side hearing aid, the data based on the datastream can be signal transmitted by the second device. Here, the right-side hearing aid receives that signal and outputs an electrical signal to a receiver (speaker) of the right-side hearing aid which is in the right ear of the recipient, thus evoking a hearing percept in the right ear based on the data contained in the MI radio signal.

In this regard, the first device does not process the data of the datastream as noted above. instead, it relies on the already processed data supplied by the MI radio link. But again, it is noted that in an exemplary embodiment, instead of an MI radio link, a Bluetooth link could be utilized between the first and second device, or any other link that can have utilitarian value.

In an exemplary embodiment, method action 1410, the action of at least one of receiving the first wireless signal or sending the second wireless signal by the first device, can includes sending the second wireless signal, wherein the second wireless signal serves a spatial functionality in the method. In this regard, as noted above, output signal 1198 from hearing aid 2420L of system 707XY can be used by the component in the environment, such as television 1280, for purposes of beamforming the outputted signal 1184 there from. Output signal 1198 thus provides locational information relating to at least one of the two hearing aids of system 707XY to television 1280. In an embodiment, the environmental component can simply be informed down the vector of the received wireless signal from the hearing aid. In this regard, the vector shown in FIG. 12 for example may not be perfectly accurate in that the beams would instead be directed towards the hearing aid that outputted the wireless signal directed to the environmental component. However, again, there could be an offset programmed into either the environmental component/a system of which that is apart, and/or the sensory supplement system. With regard to the former, the environmental component/infrastructure component could “know” to adjust the output signal slightly or more than slightly owing to the fact that the receiving hearing aid would be located 8 to 14 inches or so to one side or the other side of the origin of the wireless signal received by the environmental component. Certain things could be assumed, such as the recipient will be looking or facing towards the environmental component, and the one hearing aid will be on about the same level as the other hearing aid with respect to location in the direction of gravity, etc. Corollary to this is that in an exemplary embodiment, the transmitting hearing aid that transmits to the environmental component can provide an offset in the signal so that the environmental component will be “tricked” into beamforming the output towards not the transmitting hearing aid, but the receiving hearing aid. Still further, offset signals could be embedded in the wireless output signal to the environmental component to instruct the environmental component to alter the beamforming accordingly. Any device, system, and/or method that can accommodate the offset between the two hearing aids with respect to the output from the environmental component can be utilized in at least some exemplary embodiments providing that the art enables such. Still, as noted above, the offset might be de minimis and otherwise will not be overtly addressed in certain embodiments.

Accordingly, in an embodiment, method action 1410, the action of at least one of receiving the first wireless signal or sending the second wireless signal by the first device, includes sending the second wireless signal. In this embodiment, the second wireless signal provides (1) a vector and/or location of the first device relative to a remote device remote from the first device and the second device and/or (2) provides data indicative of a global orientation of the first device relative to the remote device remote. With regard to the former, this could be achieved by triangulation or trilaterion. This could be angle of attack or angle of departure. The vector feature indicates an orientation of a line between the two devices, whereas location indicates a three dimensional value, and thus in simplistic terms, if the vector was expressed in terms of the two angles of a spherical coordinate system, the location would provide the radius to those two angles (distance). With regard to the latter, this could be GPS data or some other coordinate data.

In this embodiment, the remote device and/or a second remote device in signal communication with the remote device streams the data stream in a specific direction relative to another direction based on the sent second wireless signal that would or might otherwise be the case based on the provided location/vector/data. This is the embodiment of FIG. 12 and FIG. 11 respectively. In an embodiment, embodiment, the remote device and/or a second remote device in signal communication with the remote device streams the data stream at a specific signal strength relative to another signal strength that would or might otherwise be the case based on the provided location/vector/data. As noted above, this can have utilitarian value with respect to avoiding interference for example. In an embodiment, embodiment, the remote device and/or a second remote device in signal communication with the remote device streams the data stream at a specific frequency relative to another frequency that would or might otherwise be the case based on the provided location/vector/data.

In an embodiment, method action 1410 includes receiving the first wireless signal, wherein the first wireless signal provides spatial information to the first device. This spatial information could be a direction and/or vector and/or location and/or global orientation data of the device that is streaming the data or a device related to such. In an embodiment, the first device provides data to the second device based on this spatial information, and the second device operates a receiver and/or transceiver thereof based on the data based on the spatial information to receive the streaming data. Accordingly, method action 1410 includes, in some embodiments, receiving the first wireless signal, and a remote device remote from the first device and the second device and/or a second remote device remote from the first device and the second device in signal communication with the remote device streams the data stream, and wherein a receiver and/or transceiver of the second device is controlled in a specific manner relative to another manner based on data based on the received first wireless signal. In an embodiment, the first wireless signal provides spatial information to the first device.

And consistent with the division of labor noted above, the first device can automatically communicate a third signal, which can be wireless or wired, depending on the embodiment, from the first device to the second device (e.g., via link 1122 or 1132, etc., which can be unidirectional or bidirectional). In this embodiment, the receiver and/or transceiver of the second device is controlled based on the third wireless signal (e.g., to focus signal capture in a given direction, such as towards TV 1180), wherein the third wireless signal includes spatial information based on the first wireless signal. The system 707XX or 707XY for example can analyze the third wireless signal to determine which direction or which frequency, etc., the receiver and/or transceiver should be set to so as to better receive the streaming data.

The above said, in another embodiment, the first device does not communicate with the second device (both ways). That is, for example, there is no MI radio link (or any other link), or at least not one that is used while the method is execute. Alternatively, the only link is a transcutaneous link where data and power is provided only from the external to the implant or, if there is back telemetry from the implant, it does not have the locationality data and/or data based on the streamed data. And this can be the case with MI radio link for the external devices (and note that MI radio can be used transcutaneously): data sent over the link one way or both ways it does not have the locationality data and/or data based on the streamed data.

As noted above, the first device can be a sensory prosthesis assistant device.

In an embodiment, there is a system, comprising a first device and a second device, wherein the system is a sensory supplement system. The devices can be any of those detailed herein and/or variations thereof and/or other devices that can enable the teachings detailed herein. More on this in a moment. However, in this exemplary embodiment, the system is such that a communication load of the system is split between the first device and the second device. This is consistent with the embodiments above where, for example, the data streaming part of the communication load is handled by one device or one component of the prostheses system and the locationality function is handled by another component or device. In this exemplary embodiment, at least one of the first device or the second device is configured to be one of worn on or implanted in a recipient of the system. By worn on a recipient, it is meant for example, a behind-the-ear device or an off the ear device that is magnetically coupled to the head of a recipient via an implanted magnet or by a device such as a soft band device that utilizes an elastic band to hold a component against the head, all by way of example only. This could be headphones or and in the ear device. This could be a watch for that matter. This is contrasted to, for example, a handheld device such as the portable assistant 2401 above or a laptop computer.

Splitting of the communication of the load corresponds to a labor split. In some embodiments, with respect to memory and/or CPU percentage usage and/or power consumed on a unit time basis, the first device bears equal to or greater than 10, 15, 20, 25, 30, 35, 40, 45, 50, 55, 60, 65, 70, 75, 80, 85, or 90%, or any value or range of values therebetween in 1% increments (e.g., 27, 33, 42-57%, etc.) of the total amount utilized by the entire system for communication. This can be all aspects of communication, or communication associated with the Bluetooth systems and/or can be the communication associated with the Bluetooth systems and the local link between one device and the other device, such as the MI radio link. In an exemplary embodiment, the aforementioned split can be based on the aspects of the system required or involved in receiving the data stream, processing the data stream, providing the data stream from one device to another, and implementing the locationality features (including developing the locationality data and/or receiving the locationality data and/or providing the locationality data and/or communicating locationality data to the other device by MI radio (for example) so the other device can adjust the receiver and/or transmitter, all depending on applicability). The idea is that no single device is bearing 100% of the communications load. This can have utilitarian value for a variety of reasons. This can ensure that one or both of the devices are not “maxed out” owing to the communications features. There are additional reasons for the utilitarian value of this that are briefly described below.

In an embodiment where, for example, the first device is configured to be one of worn on or implanted in a recipient of the system, the second device can be a hand-held system assistant (e.g., smart phone or a dedicated device) and/or body worn system assistant (smart watch for example, or a dedicated device) configured to capture a data stream from a device in an environment of the system (e.g., the television 1280 for example). And note that in some embodiments, the device in the environment of the system can be part of the system.

Consistent with the teachings above, where the first device includes at least one of a first receiver, first transmitter or first transceiver (“first” here is used simply as a nomenclature vehicle, and does not represent primacy), the second device can include at least one of a second receiver, second transmitter or second transceiver. In this exemplary embodiment, the system is at least one of configured to reversibly or irreversibly dedicate the at least one of a first receiver, first transmitter or first transceiver to spatiality functionality or configured to reversibly or irreversibly dedicate the at least one of a second receiver, second transmitter or second transceiver to audio and/or visual functionality. By reversibly dedicate, it is meant that the functionality of that device can be focused on that functionality during a first period of time and then subsequently changed to focus on another functionality at a subsequent period of time. This can be done via software and/or by control of a processor or chip or the like of one or both of the devices of the system, or could be executed by the recipient by input utilizing a switch for example. The point is, the dedicated functionality can change at a subsequent date without having to take apart the system or replace certain components for example. By rough analogy, a vehicle is configured to be reversibly placed into reverse (part in the double reversal). By irreversibly dedicate, it is meant that the functionality cannot change after its dedicated without taking apart the system or replacing certain components for example. By rough analogy, the old Sherman tank had no reverse. Once the transmission was dedicated, the tank could only go forward or be placed in neutral.

With respect to the phrase spatiality functionality, this includes any of the features detailed herein, whether based on directionality or based on a vector or based on a three-dimensional locationality system utilizing Cartesian coordinates for example etc.

Corollary to the above is that in an exemplary embodiment, the first device includes at least one of a receiver, transmitter or transceiver that is dedicated to spatiality functionality and/or the second device includes at least one of a second receiver, second transmitter or second transceiver that is dedicated to audio and/or visual functionality.

While the embodiments above have focused on the receiver transmitter and/or transceiver being dedicated, in an alternate embodiment, it can be the software stack or the Bluetooth system stack that is so dedicated. More discussion on this below. But note that in some embodiments, as will be detailed below, the stacks can be divided between the components.

In an embodiment, the communication load includes locationality and content, wherein the content is an audio, visual and/or audio/visual data stream, wherein the locationality is the responsibility of the first device and the content is the responsibility of the second device. In an embodiment, the communication load includes spatiality (which includes but does not require locationality-again, simple directionality can be used in some embodiments) and content, wherein the content is an audio, visual and/or audio/visual data stream, wherein the locationality is the responsibility of the first device and the content is the responsibility of the second device. In these embodiments, such as where the first device is configured to be one of worn on or implanted in the recipient of the system and the second device is configured to be one of worn on or implanted in the recipient of the system, the locationality and/or spatiality is dedicated to a stack of the system and the stack cannot run together with locationality and/or spatiality and content on a same receiver and/or transceiver of the first device and the stack cannot run together with locationality and content on a same receiver and/or transceiver of the second device. Further, in an embodiment, the content is dedicated to a second stack of the system and the second stack cannot run together with spatiality and content on a same receiver and/or transceiver of the first device and the second stack cannot run together with spatiality and content on a same receiver and/or transceiver of the second device.

In an embodiment, one of the two components operates an audio stack. In some embodiments, the audio stack is a feature that is utilized with streaming data. Thus, in an embodiment, the component that is dedicated to the audio and/or visual functionality would run the audio stack. That said, in an exemplary embodiment, the audio stack can be broken up between two components depending on the processing power required. In an embodiment, the concept of breaking up the stack is applicable to not just the audio stack, but any stack. The Bluetooth stack can be broken up in accordance with the teachings herein.

It is noted that embodiments include components where a given feature disclosed herein is only on/in one of the two components and/or a given feature is broken up between two or more components, unless otherwise noted, provided that the art enables such.

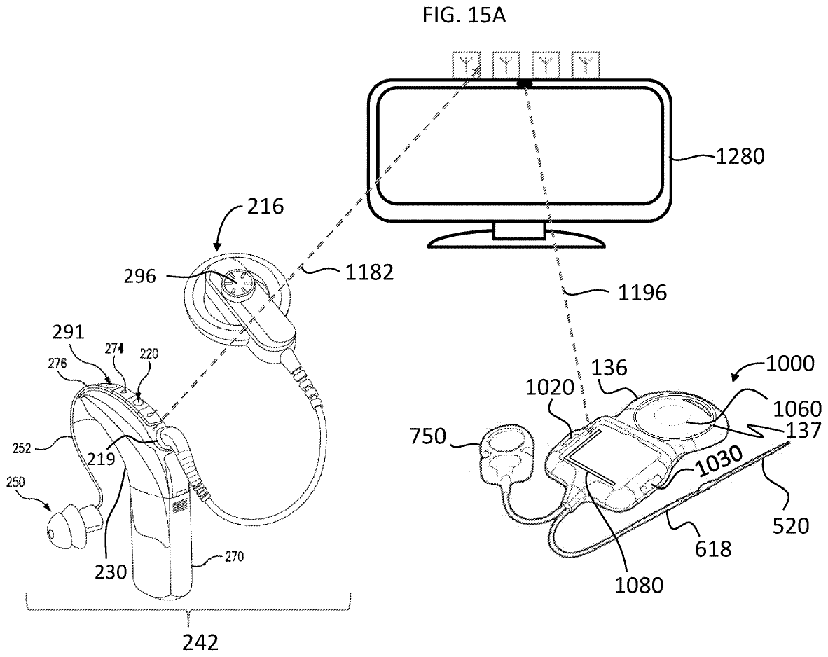

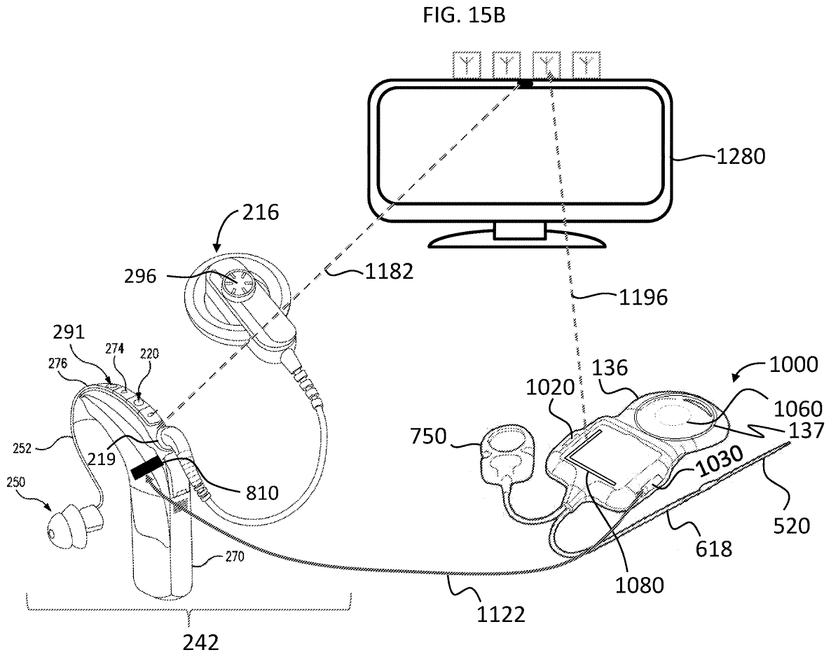

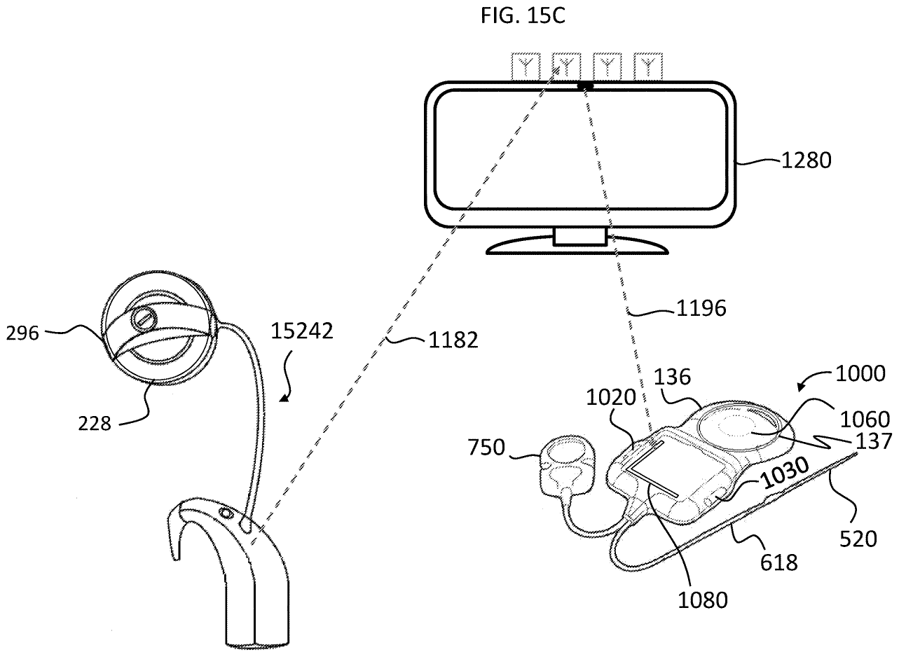

In an embodiment, the communication load includes spatiality related aspects and content related aspects, wherein the content is an audio, visual and/or audio/visual data stream. In this exemplary embodiment, the first devices configured to be worn on the recipient, and the second device is configured to be implanted in the recipient. In this exemplary embodiment, the second device is provided with a Bluetooth subsystem and is configured to receive the content. FIG. 15A depicts an external component 242 of a cochlear implant (actually, a bimodal system-in some embodiments, the external component 242 does not have acoustic hearing aid functionality, and thus there would be no in-the-ear component 250) and the implantable component of a cochlear implant 1000. Here, it is seen that the implantable component 1000 receives the wireless data stream 1196 from television 1280 by the Bluetooth antenna 1080 thereof. Also shown is that the external component is providing spatial information via wireless transmission 1182 to television 1280 (via Bluetooth antenna as well, but not shown-reference to the Bluetooth antenna and system above is made in the interest of textual economy). In an alternate embodiment, it is the first device configured to be implanted in a recipient of the system, and the second device that is configured to be worn on the recipient of the system. That is, with respect to FIG. 15B the first device is cochlear implants component 1000, and the second device is the behind-the-ear component 242, and thus the first device provides spatial data to the television 1280, and the second device receives the streaming data from television 1280.

Referring back to FIG. 15A, in an exemplary embodiment, there is no link between implantable component and the external component other than the transcutaneous link between the inductance coils thereof, which is used two power the implant, and provide in some instances data to evoke a hearing percept based on the ambient environment. This is contrasted to at least some of the embodiments disclosed above, where there is, for example, an MI radio link between the external component and the implanted component or otherwise between two components of the system, where one component provides a signal to the other component based on the processed data of the data stream. That is, in some exemplary embodiments, the external component 242 does not receive any data based on the data stream. The implanted component is configured to process the data stream and evoke a hearing percept thereon.

FIG. 15C presents another exemplary embodiment, where the prosthesis system is making full use of the fact that the implanted component 1000 is a totally implantable cochlear implant. Component 1000 includes an implantable microphone 750. Thus, in this exemplary embodiment, the implantable component does not need the external component to capture sound and/or process the captured sound (note capturing sound is different than receiving a data stream based on sound-capturing sound as used herein refers to the use of a microphone or other transducer that transduces pressure waves or the like that travel through the ambient environment and are received by the microphone for example, and transduced into an electrical signal or other output signal). Briefly, it is noted that in some embodiments, an external device that is utilized to capture sound can be used instead of relying on the implanted microphone 750, as the external device can in some instances have less attenuation in that there is not a layer of skin over the microphone. This is the scenario depicted in the exemplary embodiment of FIG. 15A and the scenario depicted in FIG. 15B, although even then, in some embodiments, it could be that the external device is simply being utilized to power the implant and/or to recharge the batteries or other power storage device of the totally implantable component 1000. In this regard, in many exemplary embodiments, there will be a need to recharge the batteries of the totally implantable component. This is achieved by the utilization of an external device such as external component 242 or external component 15242 below as will be described in more detail below. And note that the external component need not be present all the time for the implantable component to operate as a totally implantable hearing prostheses. Indeed, in many embodiments, the external component will only be present or only needs to be present for 10 or 20% of the operating time of the implantable component, because that is all the time that it takes to charge the batteries of the implantable component, and the rest of the time, the implantable component can operate autonomously without the need for power and/or being recharged from the external component. The point is that in at least some exemplary embodiments, the external component is not operating to capture in many instances of use.

In this regard, the arrangement of FIG. 15C depicts an external component 15242 that is utilized to recharge the totally implantable component 1000, and does not have sound capture features, or at least does not provide a signal to the implant based on the captured sound. This device can be used for the limited amount of time needed to recharge the batteries of the totally implantable hearing prostheses 1000. But in this exemplary embodiment, external component 15242 includes a Bluetooth antenna/system (not shown, but comparable to those above). Here, the external component 15242, which is used to recharge the implantable component and otherwise would not be used other than to do such is also used to implement the spatial functionality of the system. Here, external component 15242 is utilized to simply execute the spatial functionality of the labor split (in some embodiments, 15242 power the totally implantable hearing prostheses 1000, if it is present on the ear of the recipient, and the headpiece 296 is over the inductance coil of the implant, it can be utilitarian to simply use the external component to power the implant instead of relying on the implant's batteries, or otherwise to essentially continuously or frequently periodically recharge the batteries of the implant and thus essentially keeping them constantly “topped off”). In this exemplary embodiment, there can be a scenario where there is no data communication between the external component and the implanted component. Indeed, if the external component is not providing power to the implantable component, there would be no communication between the external component and the implantable component at all. Note further that in some embodiments, the headpiece and accompanying lead could be removed from the body of the BTE device of component 15242 so that the recipient need not have to have the headpiece located against his or her head. This can have comfort and/or aesthetic utilitarian value in some instances. In such a scenario, the BTE device would be utilized to execute the spatial functionality, and variable might not be able to be used for any other reason with the system. And note that while the embodiment of FIG. 15C shows the external component providing a signal to the device in the environment, in an alternate embodiment, the reverse can be the case with respect to executing the spatial functionality. Still, that would require some communication between the external component the implanted component to relay the data relating to spatiality to the implant so that the implant can control the Bluetooth system thereof in accordance with the teachings detailed herein.

Still, in other embodiments, external component 242 does so receive the data based on the data stream. This is depicted by way of example only with respect to data link 1122 extending from the MI radio coil 1030 of the implantable component (coil 1020 could also be used or instead could be used) to the coil 810 of the external component. Again, while the link is shown is bidirectional, in an exemplary embodiment, the link can be unidirectional. And note that while the embodiment of FIG. 15A has been shown to not have a link, in other embodiments, there can also be the MI radio link or any other link. Note also that instead of communicating over the MI radio, the Bluetooth systems of the external component and the implantable component can be utilized for communication. Additionally, while the embodiment shown depict direct communication between the external component in the implantable component, in an alternate embodiment, there could be communication through the portable assistance device, such as handheld device 2401. Thus, in an exemplary embodiment, the implantable component could communicate with the handheld device, and then the handheld device can relay that information to the external component and/or vice versa.

Note that the above arrangements can also be applicable to the totally external systems, such as a left-hand side and a right hand side conventional hearing aid system. Note further that while the embodiments above with respect to a conventional hearing aid system have been described in terms of a bilateral hearing supplement system, in other embodiments, it could be that only one side evokes a hearing percept. The other side is dedicated to simply splitting the communications load. For example, the right-hand side component may not be a hearing aid, but instead could be a device configured to solely receive the data stream and process the data stream. In another exemplary embodiment, the right-hand side component could be a device configured to solely execute the spatial functionality detailed herein. This can be also the case for the left hand side component.

In at least some embodiments, the first device includes at least one of a first receiver, first transmitter or first transceiver, the second device includes at least one of a second receiver, second transmitter or second transceiver. In some of these embodiments, the first device includes a first Bluetooth standard on an ASCI of the first device and the second device includes a second Bluetooth standard of a later origin than the first Bluetooth standard. In this regard, some embodiments will be implemented over a number of years if not decades. The implantable component will be implanted in the recipient and likely remain implanted for tens of years. The implant will not be able to be upgraded with respect to hardware thereof. In this regard, after the time of implantation, the ASICS for example, will be the technology available as of that date. Conversely, the external component, such as the behind-the-ear device or the off the ear device, will be able to be upgraded. By way of example only and not by way of limitation, 2, 3, 4, 5, 6, 7, 8, 9, or 10 years or more after implantation, a recipient may get a new external device such as a new behind-the-ear device with a new sound processor with respect to a hearing device such as a cochlear implant. This new behind-the-ear device will replace the original behind-the-ear device that was utilized with the implant. This new behind-the-ear device will be compatible with the implant. Thus, this new behind-the-ear device or otherwise this new external component will be upgraded with later versions of Bluetooth for example. It can contain a new Bluetooth chip by way of example. Conversely, the circuitry and otherwise hardware the implant will be that which was the case at year zero. The Bluetooth chip therein by way of example will be the chip that was implanted at year zero. The Bluetooth chip in the external component could be one or two or three or four or more generations advanced from that of the chip of the implant. Thus, the after mentioned embodiment where the second device includes a second Bluetooth standard of later origin contemplates this potential scenario.

In an exemplary embodiment, the first device is at least X years old, where X is 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, or any value or range of values therebetween in 0.5 increments. In an exemplary embodiment, the second device and/or one or more components associated with the communication load is less than and/or equal to Y years old, where Y is 1, 2, 3, 4, 5, 6, 7, 8, 9, or 10, or any value or range of values therebetween in 0.1 increments. In an exemplary embodiment, the Bluetooth standard or communication protocol of the first device is at least X years old, and the Bluetooth standard or communication protocol of the second device is less than and/or equal to Y years old (note the values need not be the same in the embodiments for example, the standard could be newer than the hardware, but the hardware could prevent upgrades of the standard beyond a certain point, thus the implant could be 15 years old and the standard could be 11 years old by way of example).

It is briefly noted that the utilization of the phrases first device and second device herein are for purposes of general differentiation, and are not rigidly applied. In this regard, any disclosure herein of a first device having a given feature and/or functionality corresponds to a disclosure of the second device having such feature and/or functionality, and vice versa, providing that the art enables such, unless otherwise noted. Thus, these phrases are used herein for interest of textual economy.

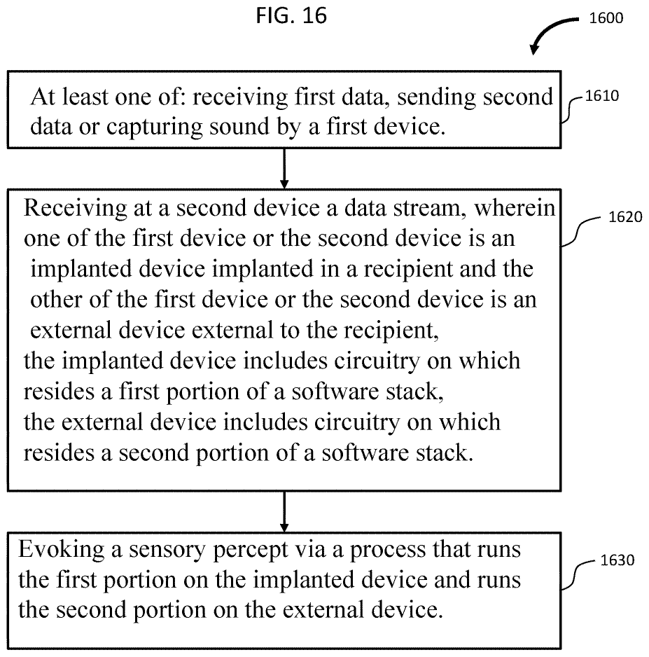

FIG. 16 presents an exemplary flowchart for an exemplary method, method 1600, which includes method action 1610, which includes at least one of: receiving first data, sending second data or capturing sound by a first device. In an exemplary embodiment, this can be executed by the left-hand or right-hand side hearing aid detailed above, or can be executed by a left-hand or right-hand side external component of an implantable prosthesis, or can be executed by an implantable component of a prosthesis, such as a so-called totally implantable cochlear implant, that includes an implanted microphone (and hence can capture sound, but also could receive first data or send second data as well, just as can be the case with the external components just noted (which includes the conventional hearing aids)).

Method 1610 further includes method action 1620, which includes the action of receiving at a second device a data stream. This can be any other of the devices just noted (or other devices, as can be the case with the first device, with the following caveat). In this exemplary method, one of the first device or the second device is an implanted device implanted in a recipient (cochlear implant, middle-ear implant, active transcutaneous bone conduction device, or retinal implant all by way of example only and not by way of limitation) and the other of the first device or the second device is an external device external to the recipient. In an embodiment, the external device is a body worn sensory prosthesis or a hand-held sensory prosthesis assistant.

In an exemplary embodiment, the first wireless signal, if received, provides spatial information to the first device related to a source of the data stream. In an exemplary embodiment, the second wireless signal, if transmitted, provides spatial information related to the second device and/or the first device to another device. This can be accomplished according to the various teachings above by way of example.

Consistent with the teachings above, the implanted device includes circuitry on which resides a first portion of a software stack, the external device includes circuitry on which resides a second portion of a software stack. Method 1600 further includes method action 1630, which comprises the action of evoking a sensory percept via a process that runs the first portion on the implanted device and runs the second portion on the external device. In this embodiment, the system stack is thus split between the two devices. The system stack could be the Bluetooth stack or otherwise the stack on which Bluetooth operates. Thus, in an exemplary embodiment, the software stack is a Bluetooth standard software stack. In an embodiment of method action 1630, the action of evoking a sensory percept via a process that runs the first portion on the implanted device is done while the second portion is run on the external device.

In an embodiment, the Bluetooth stack includes host and control programs that can be run on one of the two components or can be split between the two components. Conversely, direction finding requires lower programming power, and can be executed and is in some embodiments actually executed on a different system and/or with a different protocol. Thus, embodiments can include executing direction finding on one component, and some of the layers of the Bluetooth protocol on that same component, but not all of the layers of the Bluetooth protocol are so executed on that one component. Instead, at least some of the remainder or all of the remainder layers are executed on the other component.

In an exemplary embodiment, 1, 2, 3, 4, 5, 6 or 7 layers or any value or range of value therebetween in one increments of the Bluetooth protocol is executed on one component, and 1, 2, 3, 4, 5, 6 or 7 layers or any value or range of values therebetween in one increment of the Bluetooth protocol is executed on the other component. In an embodiment, all layers are executed on one component. In an embodiment, where the Bluetooth protocol has seven layers, it could be that five layers are run on one component and two layers a run on the other component. In an embodiment, the component that has the two layers running thereon also executes or otherwise has the spatial functionality. That said, in an embodiment, the layers may not necessarily consume equal amounts of processing power. Thus, it could be that the one or two or three layers that are most processing intensive are run on one component, and the remainder layers are run on the other component, which other component could also run the spatial functionality protocol.

In an exemplary embodiment, the bottom layers of the Bluetooth protocol are run on one component and the top layers are run on the other. In an embodiment, the layers that provide for coding and decoding and synchronization and otherwise keeping up with the buffer are run on one component, and the other layers or at least some of the other layers a run on the other component. The spatial functionality protocol is run on one of the two components.

Indeed, in an exemplary embodiment, the spatiality protocol requires less layers and there is no need for encoding and decoding.

As noted herein, there are embodiments that utilize Bluetooth direction finding. Bluetooth low energy can include Bluetooth direction finding. And note that embodiments include the utilization of Bluetooth low energy protocols. Thus, in an embodiment, one component can run the directionality layers, and the other component can run the audio layers. Corollary to this is that in some embodiments, one component handles the directionality packets and the other component handles the audio packets.

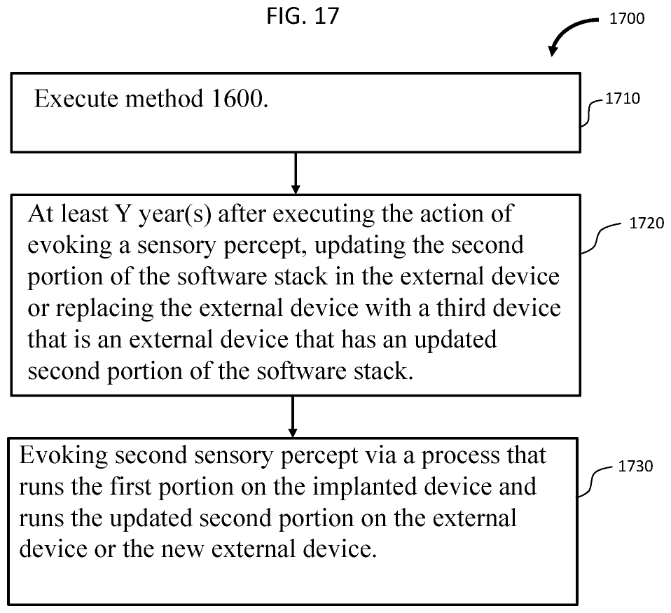

FIG. 17 includes an exemplary flowchart for an exemplary method, method 1700, according to an exemplary embodiment. Method 1700 includes method action 1710, which includes the action of executing method 1600. Method 1700 also includes method action 1720, which includes the action of at least Y year(s) (where Y can be any of the Y values above) after executing the action of evoking a sensory percept, updating the second portion of the software stack in the external device or replacing the external device with a third device that is an external device that has an updated second portion of the software stack. This corresponds to updating the communication standard, such as the Bluetooth standard, as the standard evolves and otherwise progresses. Method 1700 further includes method action 1730, which includes the action of evoking second sensory percept via a process that runs the first portion on the implanted device and runs the updated second portion on the external device or the new external device.

In an embodiment of the above method, the first portion of the software stack is an earlier version of a Bluetooth standard than the second portion of the software stack. In an embodiment, the first portion is at least Y years older than the second portion. In an exemplary embodiment, the first portion of the software stack is the latest version possible to be implemented in the implant without explanting the implant and/or developing a modified standard specifically for the implant or otherwise providing a version that is not a standard version.

FIG. 17 presents an algorithm for an exemplary method, method 1700, which includes method action 1710, which includes the action of executing method 1600. Method 1700 further includes the method action 1720, which includes the action of capturing a stream of data with the implanted device or the external device (and in some embodiments, only one or the other, but not both), the stream of data being audio, visual, and/or audio/visual data. The stream of data can be the data from television 1280, or any other environmental device which the technology detailed herein can be applicable. Method 1700 further includes the method action 1730, which includes the action of processing the stream of data utilizing the first portion and the second portion, wherein the action of evoking a sensory percept includes the action of processing the stream of data.

In view of the above, it can be seen that the spatial information obtained using the various algorithms detailed herein and/or programs herein and/or functionalities herein, can be used for beamforming of the radio wave between a transmitter and receiver. In an embodiment, this can minimize the spatial power the transmitter transmits in directions that are not in line with the receiver, reducing the power usage of the transmitter and reducing collisions in crowded areas. In an embodiment, the teachings detailed herein can result in a reduction of at least 20, 25, 30, 35, 40, 45, 50, 55, 60, 65, 70, 75, 80, 85, 90 or 95 or more percent or any value or range of values therebetween in 1% increments of the power usage of the transmitter (and/or receiver) and/or transceiver relative to that which would be the case in the absence of the teachings detailed herein, all other things being equal.

The above can also have utilitarian value with respect to selecting which transmitter / transceiver to use to transmit data to the sensory supplement system if multiple transmitting devices and/or antennas are present. Accordingly, embodiments include selecting one or more transmitters and/or receivers from a group consisting of at least more than one of the number selected based on the spatiality functions and teachings detailed herein. By way of example, if an environment includes Z transmitters that could be used to transmit to the sensory supplement system, the teachings detailed herein include scenarios where W transmitters are selected based on the spatiality functionality herein, where Z can equal 2, 3, 4, 5, 6, 7, 8, 9, 10, 11, 12, 13, 14 or 15 or more or any value or range of values in one increment and W can equal 1, 2, 3, 4, 5, 6, 7, 8, 9, 10, 11, 12, 13 or 14 or more or any value or range of values therebetween in one increment.

Embodiments can include modifying data (e.g., an acoustic signal) based on the relative position of the receiver and transmitter, adding spatial information for the listener and/or modify the acoustic signal processing parameters of the hearing aid based on its relative position to possible acoustic audio sources. In this regard, while the embodiments above have focused on applying the directionality and/or spatiality features herein towards application where there is a stream of data provided at the megahertz and/or gigahertz frequencies, in other embodiments, the quote data” can be a simple acoustic signal within the audible spectrum of 20 to 20,000 Hz. Accordingly, any teaching herein regarding utilization of the spatiality features in combination with the high-frequency data streams corresponds to an alternate disclosure of utilizing the spatiality features with ambient sound. By way of example only and not by way of limitation, data stream 1182 of FIG. 15A would instead be sound output from a speaker of the television 1180, which is captured by the sound capture device of the external component 242. In an embodiment, the component(s) can use the angle between it (them) and an acoustic audio source to direct their algorithms to this audio source, improving signal-to-noise ratio.

In view of the above, in an exemplary embodiment, there is a method that includes executing method action 1400, and also the action of capturing ambient sound with a transducer of the first device and/or the second device, and adjusting a processing algorithm used to process the captured ambient sound based on the data based on the first wireless signal and/or based on the data based on the second wireless signal. Alternatively, and/or in addition to this, beamforming of the microphones of the given device(s) can be executed in addition to this or instead of the adjustments of processing algorithm.

As noted above, embodiments include tracking the location of the recipient, or more accurately, tracking the position of the one or more components involved in the spatiality methods detailed herein. While this can be utilized with respect to the beamforming teachings herein with respect to the data stream that is streamed over the high-frequency blanks, in an alternate embodiment, this can also be utilized as a basis for adjusting the sound processor algorithms that are utilized to process the captured ambient sound within the hearing frequencies. This could provide a more realistic hearing experience relative to that which would otherwise be the case.

It is noted that any method detailed herein also corresponds to a disclosure of a device and/or system configured to execute one or more or all of the method actions detailed herein. It is further noted that any disclosure of a device and/or system detailed herein corresponds to a method of making and/or using that the device and/or system, including a method of using that device according to the functionality detailed herein. Any functionality disclosed herein also corresponds to a disclosure of a method of executing that functionality, and vice versa.

It is further noted that any disclosure of a device and/or system detailed herein also corresponds to a disclosure of otherwise providing that device and/or system.

Any feature of any embodiment can be combined with any other feature any other embodiment providing that such is enabled. Any feature of any embodiment can be explicitly excluded from utilized nation with any other feature of any embodiment herein providing that the art enables such.

It is noted that in at least some exemplary embodiments, any feature disclosed herein can be utilized in combination with any other feature disclosed herein unless otherwise specified. Accordingly, exemplary embodiments include a medical device including one or more or all of the teachings detailed herein, in any combination.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A system, comprising:
a first device; and
a second device, wherein
the first device is a component of a sensory prosthesis configured to receive a data stream and evoke a sensory percept based on the data stream, and
the second device is configured to provide spatial output to the first device and/or another device remote from the second device.

2. The system of claim 1, wherein:
the first device is a hearing prosthesis component, and the data stream is an audio stream.

3. The system of claim 1, wherein:
the second device is configured to provide the spatial output the first device; and
the first device is configured to control a directionality feature of a receiver and/or transceiver based on the spatial output.

4. The system of claim 1, further comprising:
the another device, wherein
the second device is configured to provide the spatial output to the another device; and
the another device is configured to control and/or provide data for control of a directionality feature of a transmitter and/or transceiver that transmits the data stream based on the spatial output so that the data stream is directed more towards the second device than that which would have been the case in the absence of the provided spatial output.

5. The system of claim 4, wherein:
the another device includes the transmitter and/or transceiver.

6. The system of claim 4, wherein:
the transmitter and/or transceiver is part of a device separate from the another device.

7-8. (canceled)

9. The system of claim 1, wherein:
the first device is a conventional acoustic hearing aid; and
the second device is a second acoustic conventional hearing aid.

10. A method, comprising:
at least one of receiving a first wireless signal or sending second wireless signal by a first device;
receiving at a second device a data stream, wherein the second device is a component of a sensory prosthesis; and
transmitting by the second device to the first device data based on the data stream, wherein
at least one of:
a receiver and/or transceiver of the second device is adjusted based on data based on the first wireless signal, which receiver and/or transceiver receives the data stream; or
a transmitter and/or transceiver of another device is adjusted based on data based on the second wireless signal, wherein the transmitter and/or transceiver transmits the data stream.

11. The method of claim 10, wherein:
the first device does not receive the data stream; and
the method further comprises receiving by the first device the data based on the data stream, wherein the first device evokes a sensory prosthesis based on the received data based on the data stream.

12. The method of claim 10, wherein:
the action of at least one of receiving the first wireless signal or sending the second wireless signal by the first device includes sending the second wireless signal, wherein the second wireless signal serves a spatial functionality.

13. (canceled)

14. The method of claim 10, wherein:
the action of at least one of receiving the first wireless signal or sending the second wireless signal by the first device includes receiving the first wireless signal, wherein the first wireless signal provides spatial information to the first device.

15. (canceled)

16. The method of claim 10, wherein:
the action of at least one of receiving the first wireless signal or sending the second wireless signal by the first device includes receiving the first wireless signal, wherein a remote device remote from the first device and the second device and/or a second remote device remote from the first device and the second device in signal communication with the remote device streams the data stream, and wherein a receiver and/or transceiver of the second device is controlled in a specific manner relative to another manner based on data based on the received first wireless signal.

17. (canceled)

18. The method of claim 16, wherein:
the first device is a sensory prosthesis assistant device.

19. The method of claim 10, wherein:
the first device is not communicating with the second device.

20. (canceled)

21. A system, comprising:
a first device; and
a second device, wherein
the system is a sensory supplement system,
a communication load of the system is split between the first device and the second device, and
at least one of the first device or the second device is configured to be one of worn on or implanted in a recipient of the system.

22. The system of claim 21, wherein:
the first device is configured to be one of worn on or implanted in a recipient of the system; and
the second device is a hand-held and/or body worn system assistant configured to capture a data stream from a device in an environment of the system.

23. The system of claim 21, wherein:
the first device includes at least one of a first receiver, first transmitter or first transceiver;
the second device includes at least one of a second receiver, second transmitter or second transceiver; and
the system is at least one of:
configured to reversibly or irreversibly dedicate the at least one of a first receiver, first transmitter or first transceiver to spatiality functionality; or
configured to reversibly or irreversibly dedicate the at least one of a second receiver, second transmitter or second transceiver to audio and/or visual functionality.

24. The system of claim 21, wherein:
the first device includes at least one of a receiver, transmitter or transceiver that is dedicated to spatiality functionality.

25. The system of claim 24, wherein:
the second device includes at least one of a second receiver, second transmitter or second transceiver that is dedicated to audio and/or visual functionality.

26-27. (canceled)

28. The system of claim 21, wherein:
the communication load includes locationality and content, wherein the content is an audio, visual and/or audio/visual data stream;
the first device is configured to be worn on the recipient;
the second device is configured to be implanted in the recipient;
the second device is provided with a Bluetooth subsystem and is configured to receive the content.

29-40. (canceled)
